(19) United States
(12) Patent Application Publication
(10) Pub. No.: US 2016/0354223 A1
(43) Pub. Date: Dec. 8, 2016

(54) DEVICES AND METHODS FOR FACILITATING PATIENT COMPLIANCE WITH THERAPEUTIC PROCEDURES

(71) Applicant: Boa Technology, Inc., Denver, CO (US)

(72) Inventors: Robert Burns, Denver, CO (US); James Capra, Steamboat Springs, CO (US); Jesse Cotterman, Evergreen, CO (US); Michael Nickel, Golden, CO (US); William Furman O'Dell, Evergreen, CO (US); Mark Soderberg, Conifer, CO (US); Aaron Ventruini, Denver, CO (US)

(73) Assignee: Boa Technology, Inc., Denver, CO (US)

(21) Appl. No.: 15/173,133

(22) Filed: Jun. 3, 2016

Related U.S. Application Data
(60) Provisional application No. 62/170,614, filed on Jun. 3, 2015.

(51) Int. Cl. A61F 5/01; A44B 11/06

(52) U.S. Cl. CPC A61F 5/0102; A61F 5/0123; A44B 11/065

(57) ABSTRACT

A device for tightening an article about a limb includes a tension member that is guided about the article and that is tensionable to close or tighten the article about the limb. The device also includes a tightening mechanism that is configured to tension the tension member to close or tighten the article and that is further configured to maintain the tension of the tension member so that the article remains closed or tightened about the limb. The device further includes a tension limiting mechanism that prevents loosening of the tension member's tension beyond a determined threshold so that the article remains closed or tightened about the limb.

### CROSS-REFERENCE TO RELATED APPLICATIONS

This application claims priority to Provisional U.S. Patent Application No. 62/170,614 filed Jun. 3, 2015, entitled “Devices and Methods for Facilitating Patient Compliance with Therapeutic Procedures,” the entire disclosure of which is hereby incorporated by reference, for all purposes, as if fully set forth herein.

### BACKGROUND OF THE INVENTION

This invention relates generally to braces and other orthopedic devices and more particularly to closure devices that may be used with such devices.

Braces or other orthopedic devices are typically worn to support and/or provide a therapeutic effect to a limb. For optimal therapeutic results, it is preferred that such devices be properly worn for a prescribed period of time. Conventional devices may feel uncomfortable and/or unnatural to the wearer due to the inability of such devices to adjust to accommodate various needs and/or conditions. For example, most braces are unable to adjust when a wearer experiences edema or atrophy. As such, a brace that was properly fit about the wearer's limb may feel unnecessarily tight or loose when the wearer experiences either of these conditions. In some instances, the wearer may remove the brace to relief the discomfort. Removing the brace, however, negates the beneficial effects that the brace is implemented to provide.

### BRIEF DESCRIPTION OF THE INVENTION

According to one aspect, a device for tightening an article about a limb includes a tension member that is guided about the article and that is tensionable to close or tighten the article about the limb. The device also includes a tightening mechanism that is configured to tension the tension member to close or tighten the article and that is further configured to maintain the tension of the tension member so that the article remains closed or tightened about the limb. The device further includes a tension limiting mechanism that prevents loosening of the tension member's tension beyond a determined threshold such that the article remains closed or tightened about the limb.

In some instances, the tension limiting mechanism is operably coupled with a rotatable knob of the tightening mechanism. In such instances, the tension limiting mechanism may be engageable with a stop member to prevent rotation of the knob. In a specific embodiment, the tension limiting mechanism is a boss or post and the stop member is a channel within which the boss or post is disposed.

In other instances, the tension limiting mechanism is an additional tension member that is windable about a spool component of the tightening mechanism. In such instances, the additional tension member may be configured to prevent rotation of the spool component after a defined amount of the additional tension member is wound about the spool component. In yet other instances, the tension limiting mechanism is a crimp member that is attached to the tension member. In such instances, the crimp member may be engagable with a stop member after a defined amount of the tension member is wound about a spool component of the tightening mechanism to prevent rotation of the spool component. In yet other instances, the tightening mechanism is a reel based device that is operably coupled with the tension member so that operation of the tightening mechanism tensions the tension member and the tension limiting mechanism is coupled with a portion of the tension member that is remotely positioned from the tightening mechanism.

According to another aspect, a method for tightening an article about a limb includes providing an article with a device that is configured to tighten the article about the limb. The device includes: a tension member that is guided about the article, a tightening mechanism that is configured to tension the tension member and to maintain the tension of the tension member, and a tension limiting mechanism that prevents loosening of the tension member's tension article beyond a determined threshold. The method also includes placing the article about the limb and tensioning the tension member via the tightening mechanism to close or tighten the article about the limb. The method further includes restricting loosening of the tension member's tension via the tension limiting mechanism so that the article remains closed or tightened about the limb.

### BRIEF DESCRIPTION OF THE DRAWINGS

The present invention is described in conjunction with the appended figures:

FIGS. 1-3 illustrate orthopedic braces that include reel based closure devices in accordance with the embodiments described herein.

FIG. 4 illustrates the ability of a brace as described herein to be fit and adjust about a conically shaped object.

FIGS. 5a-6c illustrate embodiments of reel systems and reel system components that may be employed with a brace to ensure that an individual does not remove the brace from about a limb.

FIGS. 7a-8g illustrate various embodiments of reel systems that may be employed with a brace to enable an individual to tighten or loosen the brace within a defined range.

FIGS. 9a-9b illustrate various guide members that may be employed with a brace to enable an individual to tighten or loosen the brace within a defined range.

FIGS. 10a-10e illustrate additional embodiments of reel systems that may be employed with a brace to enable an individual to tighten or loosen the brace within a defined range.

FIGS. 11a-11d illustrate embodiments of guide members that are lockable about a brace.

FIGS. 12a-12c illustrate various embodiments of adjusting a stiffness of a brace.

FIG. 13 illustrates an embodiment of a motorized closure device that may be used to set and or adjust a tension of a brace.

FIG. 14 illustrates a brace being used to provide physical therapy by allowing the patient to repeatedly flex a body part.

FIGS. 15a-15d illustrate various embodiments of mechanisms that may be employed to indicate an applied level of tension.

FIGS. 16a-16c illustrate embodiments of a sleeve that may positioned over a bivalve cast and used to adjust the fit of the cast.

In the appended figures, similar components and/or features may have the same numerical reference label. Further, various components of the same type may be distinguished by following the reference label by a letter that distinguishes among the similar components and/or features. If only the first numerical reference label is used in the specification, the description is applicable to any one of the similar components and/or features having the same first numerical reference label irrespective of the letter suffix.

### DETAILED DESCRIPTION OF THE INVENTION

The ensuing description provides exemplary embodiments only, and is not intended to limit the scope, applicability or configuration of the disclosure. Rather, the ensuing description of the exemplary embodiments will provide those skilled in the art with an enabling description for implementing one or more exemplary embodiments. It being understood that various changes may be made in the function and arrangement of elements without departing from the spirit and scope of the invention as set forth in the appended claims.

The embodiments described herein provide devices and systems that help ensure a patient's compliance in properly wearing a medical brace or product. Medical braces or products work best when they are properly worn for a prescribed period of time. A problem with some medical braces is that they feel uncomfortable and/or unnatural to the wearer, or they are otherwise unattractive. For instance, the brace may feel too tight or may interfere with the patient's ability to perform an action, such as sleeping. For these and other reasons, patients may be inclined to remove the brace or not wear the brace properly. Removing the brace or not wearing the brace properly can be detrimental to recovery of the patient, and specifically to a body part that is supported by the brace.

The braces and products described herein help ensure a patient's compliance with wearing a brace or product in a prescribed manner and/or for a prescribed period of time. This compliance is ensured by providing brace closure devices that are not removable by the patient and/or that provide a means of detecting when the brace has been removed. In some embodiments, the brace closure device may allow the patient to adjust the fit of the brace without allowing the patient to fully remove the brace. Such closure device may allow the user to tailor or adjust the fit and/or feel of the brace to reduce or eliminate any discomfort associated with wearing the brace. The ability to tailor the fit and/or feel of the brace may encourage the patient to wear the brace in the prescribed manner and for the prescribed amount of time. In some embodiments, the amount of tension applied to close the brace may be displayed to the patient and/or physician to help ensure that the brace is not overly tight or overly loose. In some embodiments, the closure device, and thus the brace, may help with a prescribed physical therapy procedure.

Ensuring a patient's compliance with wearing a brace may provide several benefits. For example, if a physician knows that a brace will be worn in a prescribed manner and for a prescribed amount of time, the physician may be able to more specifically design a treatment plan to the individual patient. Further, the patient may experience improved healing by fully complying with a recommended treatment plan. Moreover, follow-up visits to the physician may be reduced since the physician can be assured that the brace will be worn properly. The net result of these and other benefits is increased savings in time and money.

For convenience, the disclosure will focus mainly on braces, although it should be realized that the embodiments described herein (i.e., the closure device and/or other devices) may be used with virtually any type of medical or non-medical product, apparel, garment, or other structure. For example, the embodiments (i.e., closure and other devices) may be used on various medical and non-medical shoes, boots, gloves, hats, and the like.

In addition, for convenience in describing the embodiments, the disclosure generally describes the devices, or components thereof, being closed via a reel or dial mechanism. The reel or dial mechanism typically closes the device, or components thereof, by tensioning a lace. As described herein, the dial is typically twisted to wind a lace into a spool. However, although the disclosure generally describes the closure devices, or components thereof, using a reel or dial mechanism, it should be realized that any tightening mechanism may be used and the disclosure is not limited to use of a reel or dial based device.

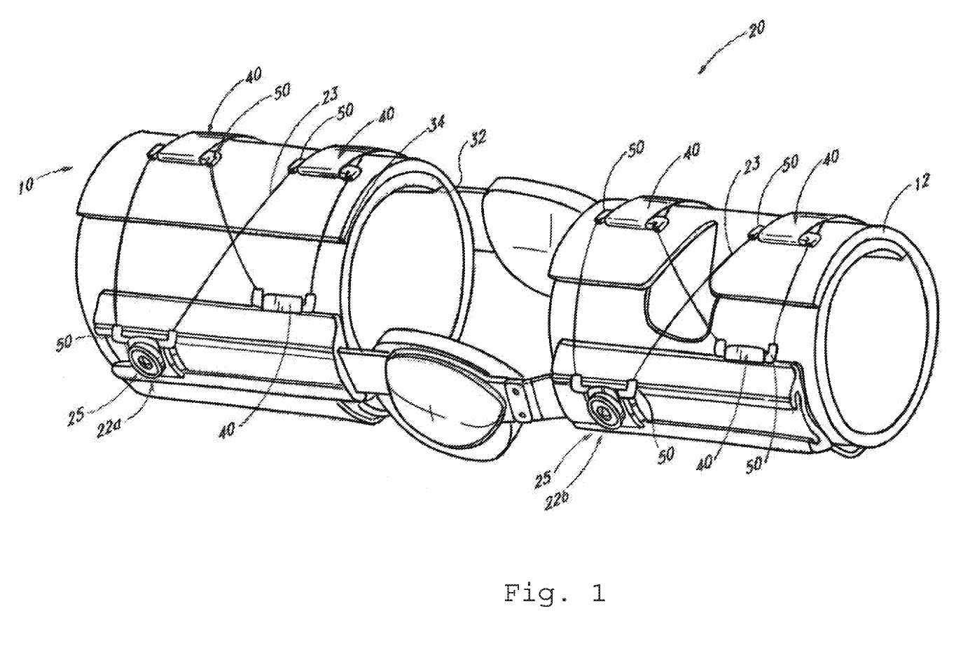

Before describing specific details of the various embodiments, a general description of a brace and closure system will be provided. Referring to FIG. 1, illustrated is an embodiment of an orthopedic brace 20. The orthopedic brace 20 illustrated comprises a knee brace that is tightened around a wearer's leg such that the knee brace substantially surrounds and protects the wearer's knee. Brace 20 may be tightened using a lacing configuration comprising two lacing systems 22a, 22b. The orthopedic brace of the illustrated embodiment is particularly concerned with relieving and/or supporting the knee joint. Although this illustrated embodiment shows the lacing systems applied to knee braces, it is to be understood that the principles discussed herein are readily applicable to any of a variety of orthopedic braces, including ankle braces, wrist braces, foot braces, elbow braces and many other types of orthopedic braces.

In some embodiments, the lacing configuration of closure system comprises two distinct lacing systems 22a, 22b. In some embodiments, each lacing system 22 includes a lace or cable 23 that is threaded through portions of the orthopedic brace and attached at opposite ends to a tightening mechanism 25 or reel, which includes a control such as a lever, crank or knob, which can be manipulated to retract the lace 23. In addition, the tightening mechanism 25 may comprises a mechanism of release, such as a button or lever, for disengaging the tightening mechanism 25, to permit the lace 23 to be withdrawn freely. In other embodiments, the tightening mechanism or reel 25 may be pulled upward to allow an internal spool to spin and the lace to be pulled freely. In yet another embodiment, the tightening mechanism or reel 25 may be unwound (e.g., counterclockwise) to release the spool and allow the lace to be pulled, or to unwind the lace. As shown in FIG. 1, the lace 23 may be threaded in a crossing pattern along a generally forward-facing portion of the brace 20, between two generally parallel rows of side retaining members or straps 40. In another embodiment, the lace 23 may be threaded or run horizontally across the brace 20. The straps 40 may consist of a strip of material attached to the brace 20 so as to define a space in which guides 50 are positioned. The lace 23 slides through the guides 50 during tightening and untightening of the lace 23. A more thorough description of the brace 20 and lacing systems, 22a & 22b, is provided in U.S. Pat. No. 8,277,401, the entire disclosure of which is incorporated by reference herein.

The orthopedic brace 20 shown in FIG. 1 is constructed to fit a wearer's leg. The upper cuff 10 is formed to fit the wearer's thigh and curves around the thigh, generally conforming to the wearer's musculature. The lower cuff 12 is similar in construction to the upper cuff 10, and is formed to fit and curve around the wearer's calf. In some embodiments, the upper and lower cuffs 10, 12 are formed from a relatively lightweight, breathable material. In some embodiments, the cuffs 10, 12 are manufactured from a cloth, fabric, or foam-like material, or a thermoformable or non-thermoformable plastic material.

As shown, each of the cuffs 10, 12 are generally formed from a single piece of material that is wrapped around itself, forming two ends 32, 34 that are drawn towards each other and, in fact, overlap. Although the ends 32, 34 are shown in an overlapping position, it should be understood that these ends might also be sized to be separated by some distance when the orthopedic brace 20 is tightened. Generally, the lace 23 may be tensioned to draw the ends 32, 34 past each other and thereby tighten the orthopedic brace 20 about the wearer's limbs. The two ends 32, 34 of brace 20 are designed to be open and fit about a patient's leg. The two ends 32, 34 are then positioned over the leg and brace 20 is tightened as described above.

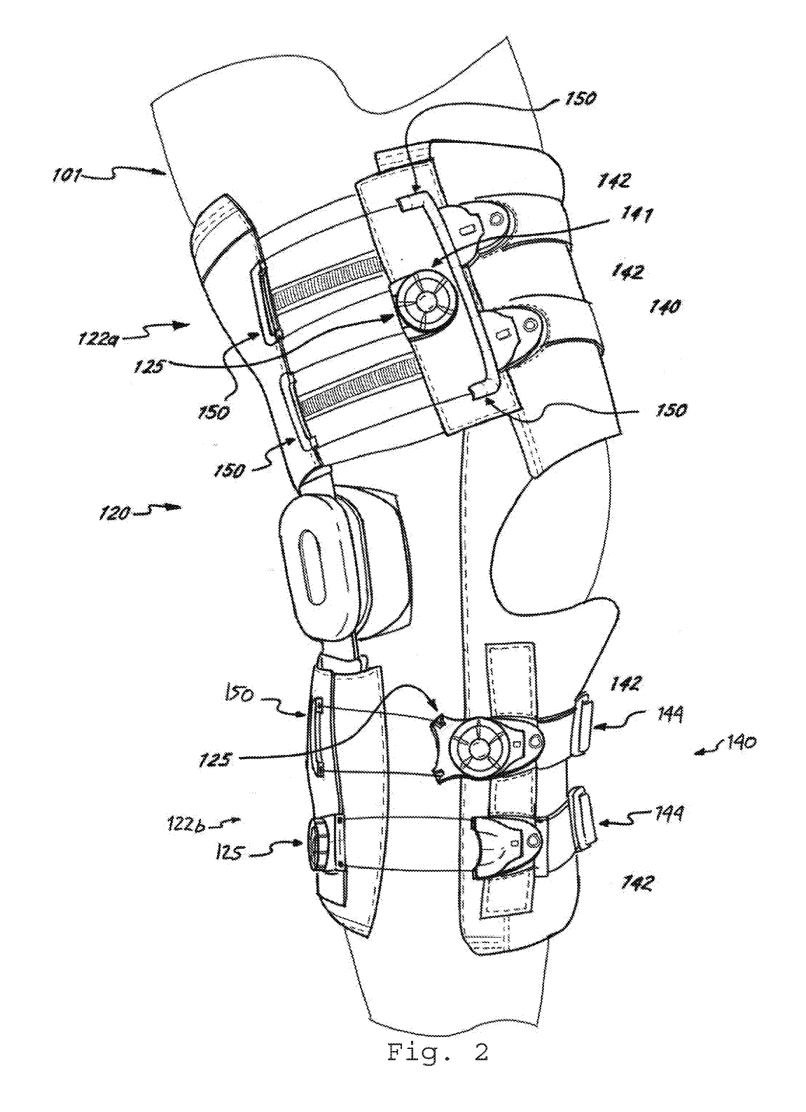

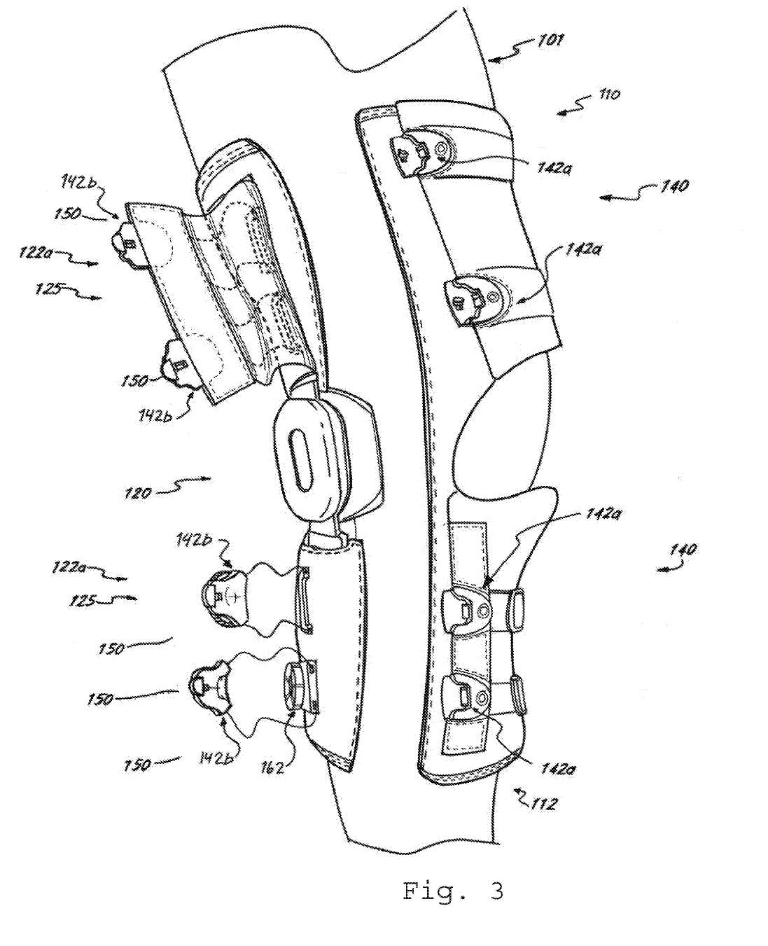

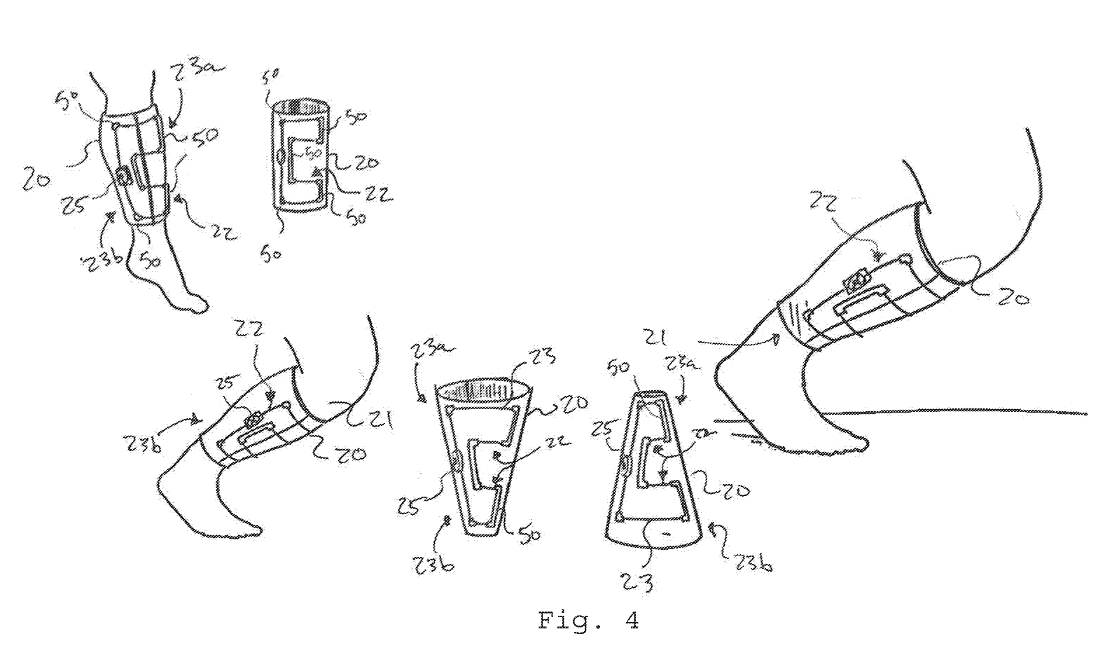

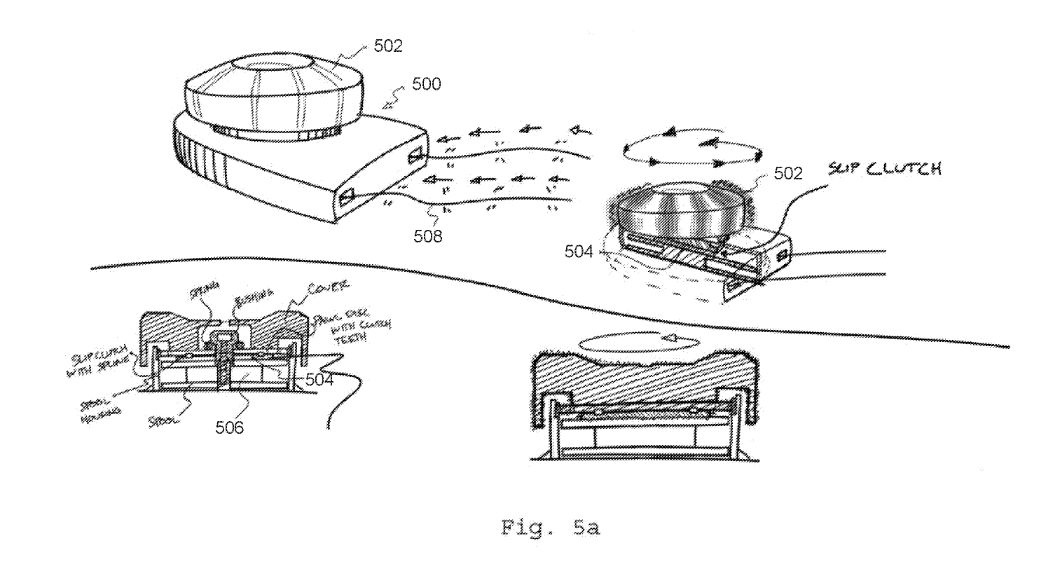

FIGS. 2 and 3 illustrate another brace 120 being fit over a wearer's leg 101. Brace 120 includes a closure system (e.g., 122a and 122b) that is described in more detail in U.S. Pat. No. 8,277,401 incorporated by reference herein. Brace 120 also includes a rough adjustment feature that permits further opening of the brace 120 to facilitate attachment of the brace 120 to a wearer's leg 101, while still providing the tightening mechanism 125 for final tightening. The rough adjustment feature may be variable length retaining members 140 that allow brace 120 to fit a wider variety of wearers' legs. In one embodiment, the variable length retaining member 140 includes adjustable straps. In other embodiments, a panel 141 may be used. The panel 141 may be coupled with a tightening mechanism 125, such as a reel and lace, to provide gross or macro adjustment of the brace 120. In some embodiments, retaining members 140 are configured to be releasably engaged with guides 150 opposite the tightening mechanism 125. The engagement may be by way of a quick release mechanism 142, such as the detachable guides described herein. In other embodiments, Fastex® buckles (shown), Velcro® or other similar mechanisms may be used. As shown in greater detail in FIG. 3, each quick release mechanism 142 may include a female component 142a and a male component 142b that are coupled over the wearer's leg 101 to allow brace 120 to be donned and doffed. Exemplary embodiments of male and female components, 142b and 142a, are described herein. In some embodiments, the female component 142 a may be attached to the guide 150 while the male component 142b is attached to the retaining member, though the arrangement of components may be switched as needed. The opposite end of the retaining member 140 may be attached to the brace such that tension in the lacing system 122 causes tension on the retaining member 140 when the quick released mechanism 142 is engaged, thereby compressing the cuffs around the wearer's limb.

Closure system 122 may include additional gross adjustment features in combination with the quick release mechanism 142 to provide a rough or gross adjustment of the closing pressure of the brace 120 prior to use of the tightening mechanism 125. For example, the closure system 122 may include ladder locks 144 (e.g., Fastex Slider®) which allow the retaining members 140 to be lengthened or shortened as needed. Though shown with two retaining members 140, in some embodiments three, four, five, six or more retaining members 140 may be employed.

FIG. 3 shows one embodiment of the brace 120 in a partially open configuration. The quick release mechanism 142 have been disconnected leaving the guides 150 attached to the brace and releasing one end of the retaining member 140. To remove the brace 120, the user may then open the cuffs 110, 112 and slide the brace from the user's leg 101. Prior to releasing the quick release mechanism 142, the user may release tension in the closure system 122 by releasing the tightening mechanisms 125 by, for example, pulling outwards on the knobs 162. Alternatively, the user may release the tightening mechanisms 125 after releasing the quick release mechanism 142 to facilitate reattachment of the brace 120 by providing additional slack in the system without adjusting the retaining members 140 themselves.

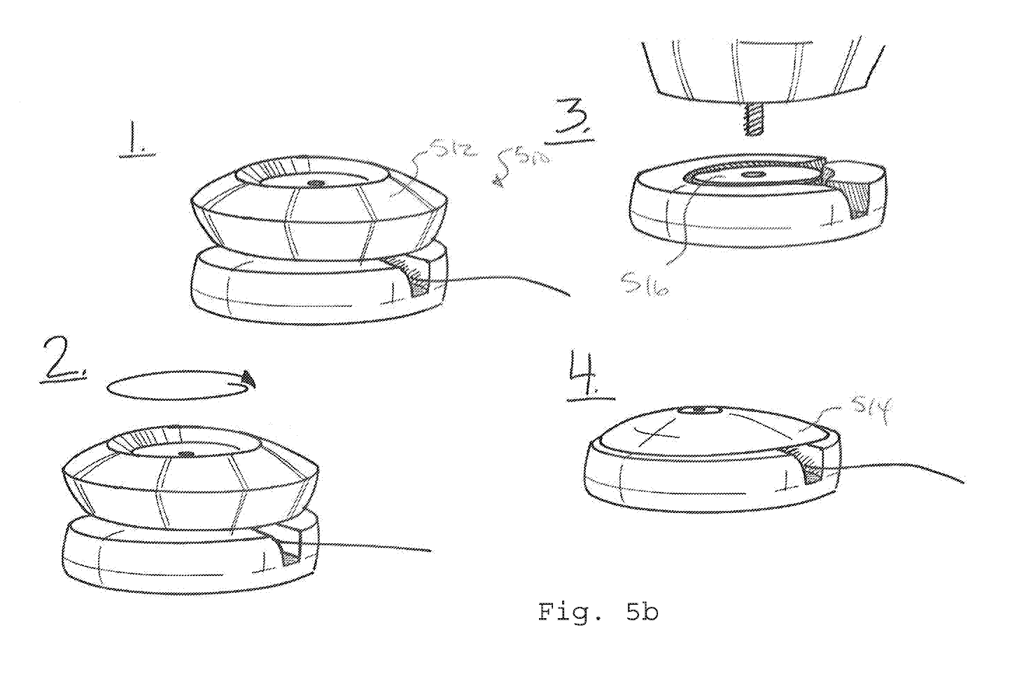

As shown in FIG. 4, one advantage of using the above described brace 20 is the increased ability of the brace 20 to fit a conical or generally cylindrical shape, such as a leg 21, arm, or any other body part of the patient. The ability of the brace 20 to fit a conical shape is provided by the lace winding system 22. As the brace 20 is fit about a conical shape (e.g., the leg 21) and the lace 23 wound via the reel 25, an upper portion 23a of the brace 20 contacts the conically shaped object. As the lace 23 is wound, the lace 23 adjusts until the lower portion 23b of the brace 20 also contacts the conically shaped object (e.g., the leg 21). Additional winding of the lace 23 will result in an approximately equal tension throughout the lace 23, which provides a relative even pressure on the conically shaped object.

Similarly, the brace 20 is able to adjust to changes in the shape of the object, such as changes in the shape of a leg 21 (or other body part) due to flexing and/or relaxing of the muscle. For example, as the leg 21 is flexed and assumes a more cylindrical shape, the lace 23 is able to slide within, or relative to, the guides 50 so that a bottom portion 23b of the brace opens or widens as a top portion 23a contracts or shrinks. Conventional braces typically do not adjust in this manner and as such, when a patient flexes his or her leg 21 (or other body part) the brace 20 is typically forced to move or migrate about the body pary, such as downward against the knee or ankle. In the embodiments described herein, because the lace 23 is able to slide relative to the brace 20 and guides 50, and the brace 20 is able to adjust to changes in shape, the fit or hold of the brace about the body part is increased and migration of the brace 20 is limited or eliminated.

As described above, one difficulty with medical braces is ensuring that a patient will not remove the brace and/or loosen or tighten the brace too much. This is a common problem with conventional braces that use Velcro® straps because the straps may be easily undone and the brace removed when the brace feels uncomfortable or is otherwise inconvenient to wear. Further, patients may be less than truthful when reporting their compliance in wearing the brace to their physician. As a result, the physician may not know exactly when and how the patient wore the brace and for how long. The embodiments described herein below reduce or eliminate some of these problems in ensuring a patient's compliance in wearing a brace.

In some embodiments, a patient's compliance in wearing a brace for a prescribed amount of time and with a prescribed tightness or fit may be ensured by providing a closure device that is not adjustable by the patient. Stated differently, the closure device, or a component thereof, may be designed so that the device is only adjustable by the physician. Thus, the physician may set the fit of the brace and the patient may be essentially forced to wear the brace as set by the physician until the physician removes or adjusts the brace.

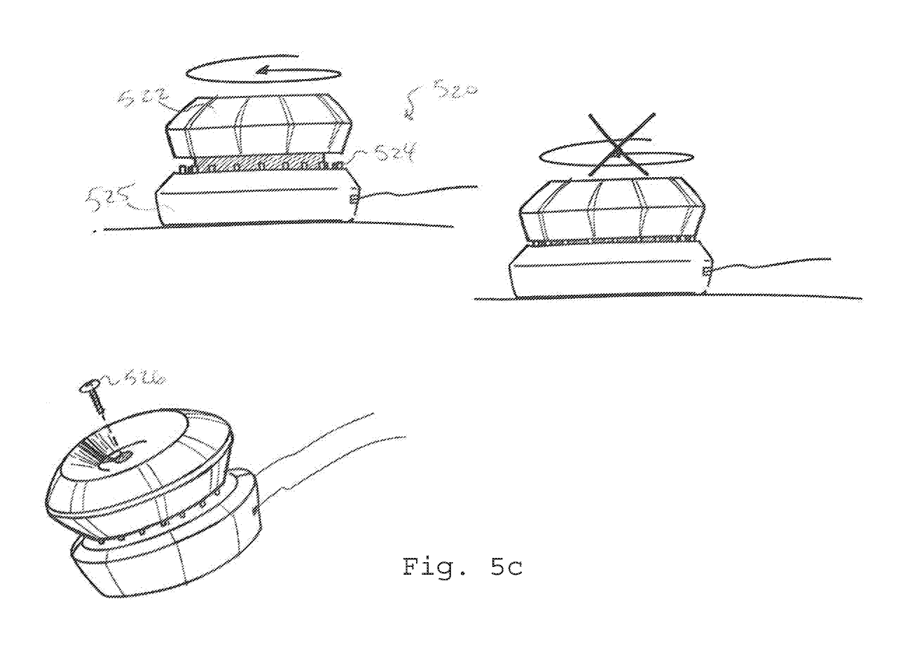

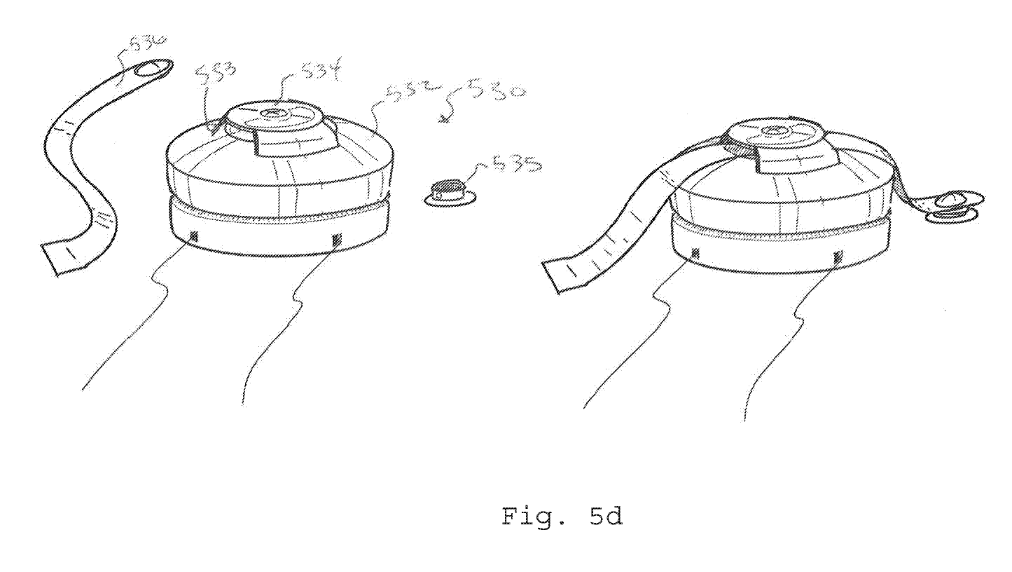

Referring now to FIG. 5a, illustrated is an embodiment of a reel system 500 that may be configured so that the patient cannot adjust the fit of the brace, or so that the patient may adjust the fit of the brace within a prescribed range. This feature of reel system 500 is provided by a slip clutch 504 that is designed to “slip” or not engage a spool 506 when a dial 502 is twisted to further tension or loosen lace of the reel system. Reel system 500 is especially good at limiting the patient from further tensioning the lace, but may also be used to limit the patient's ability to loosen the lace. In some embodiments, teeth (not shown) that typically engage at the interface 504 between the dial 502 and spool 506 may slip or slide relative to one another when the patient twists the dial 502. In some embodiments, the physician may set the slip point (i.e., torque limit) of the slip clutch.

Referring now to FIG. 5b, illustrated is another embodiment 510 of a means for eliminating a patient's ability to adjust tension in lace of a reel system. Specifically, the physician may set the tension in the system via a reel or dial 512, which may then be removed from a housing 516 by the physician, and replaced with a cap 514 or cover. The cap 514 may prevent the patient from tightening or loosening the lace of the system. FIG. 5c illustrates a similar embodiment 520. In this embodiment 520, a dial 522 is used by a physician to tension lace of the system to close and tension the brace. The dial 522 is then locked to the housing 525 to prevent the patient from further tightening or loosening the lace tension. In one embodiment, the dial 522 is locked to the housing by pressing the dial axially downward so that teeth 524 of the housing 525 engage with the dial 522. The dial 522 and housing are fixedly coupled in this configuration with a fixing component 526, such as a T-6 screw, a custom tamper proof screw, and the like.

FIG. 5d illustrates another embodiment 530 of locking a dial 532 of a reel system. Specifically, the dial 532 includes a slot 533 positioned near its top portion 534 through which a strap 536 is inserted. The strap 536 is coupled with the brace or reel system so that the strap 536 extends across the dial 532 and locks the dial in position. The strap 536 may be fixedly coupled with the brace or reel system using a tamper proof and non-removable snap, button, rivet, clamp, adhesive, weld, and the like. FIG. 5e illustrates yet another embodiment 540 of locking a dial 542 of a reel system. Specifically, a lockable cap 544 may be positioned over the dial 542 after a physician uses the dial 542 to tension the lace of the system. The cap 544 may have a hook 546 or engaging portion that snaps into or around a recess of lipped portion of the dial 542. The cap 544 may alternatively or additionally fit over a post 547 of the housing of the reel system to properly align and secure the cap 544 in place over the dial 542. The cap 544 prevents the patient from accessing and operating the dial 542 to tighten or loosen the lace. The cap 544 includes an integrated mechanism that allow the cap to be removed by depressing a detent that locks the cap down.

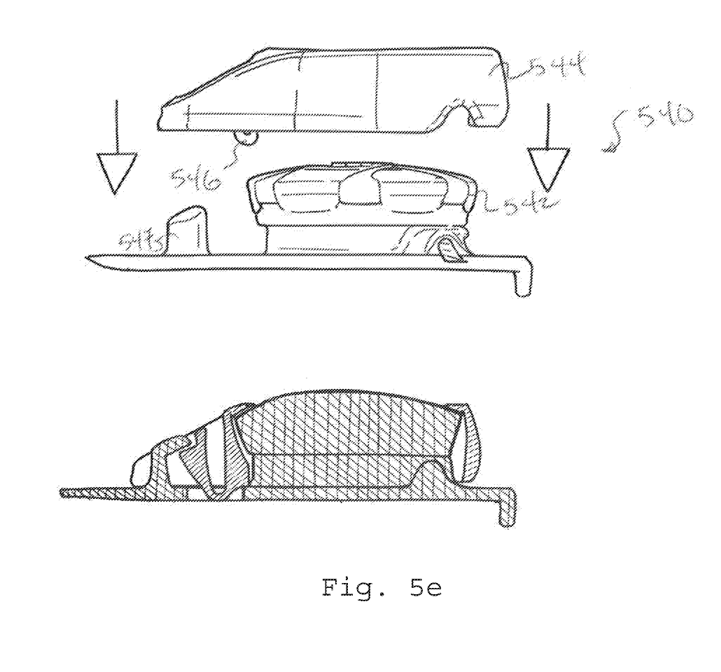

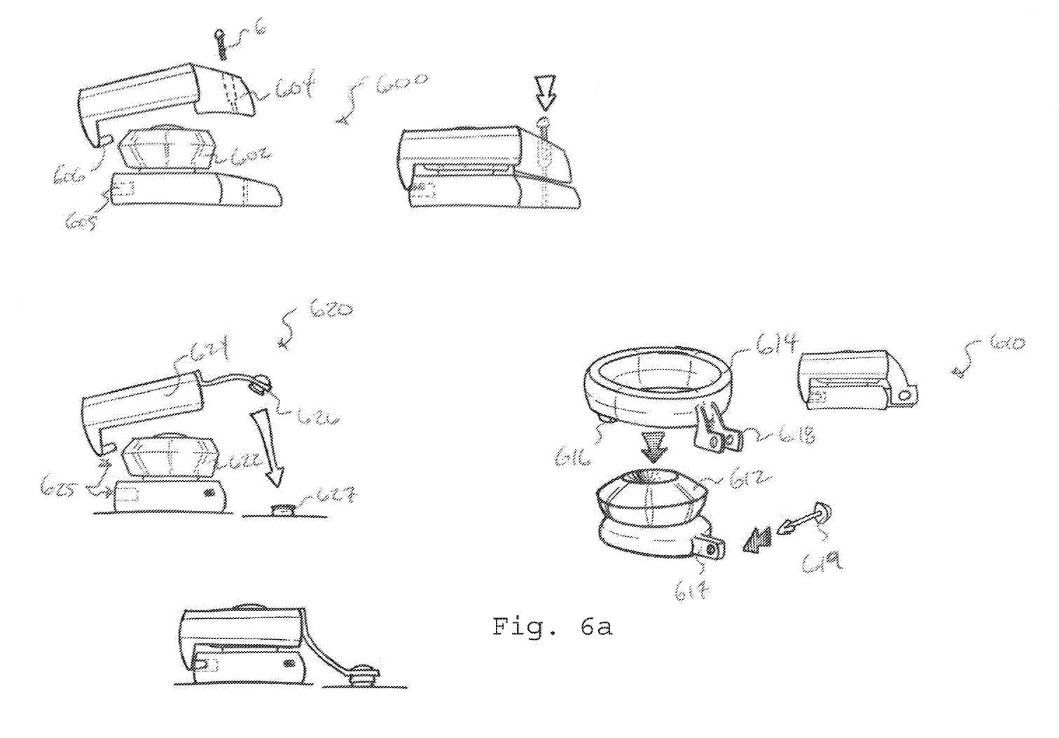

In some embodiments, the compliance with wearing a brace may be visually indicated to the physician. Stated differently, the brace or closure device may be configured so that any tampering of the closure device may be evident to the physician. Referring now to FIG. 6a, illustrated is one embodiment in which tampering of the closure device may be evident. Specifically, in embodiment 620, a cap or cover 624 may be fit over the dial 622 as described above to limit access to the dial. The cover 624 may be fixedly coupled with the brace or closure device 627, such as by using an engagement mechanism 625 and a snap 626 as described above. To remove the brace and/or loosen/tighten the lace, the strap 626 may have to be cut. Tampering of the brace will be evident if the strap 626 is cut and/or disfigured.

Embodiment 600 and 610 illustrate additional caps that may be used to cover a dial and prevent access to the dial. Specifically, embodiment 600 illustrates a cover 604 that is fit over a dial 602 and coupled therewith by inserting a tab 606 of the cover 604 within a recess of the housing. A pin, post, or screw 607 may then be inserted with an aperture of the cover 604 and housing or brace to couple the cover 604 over the dial 602. In some embodiments, the pin, post, or screw 607 may have a custom head design that allows the pin, post, or screw 607 to be removed only by a special tool. Embodiment 610 illustrates a ring 614 that is inserted over and around the dial 612 to prevent access to the dial. A post 616 or engagement feature of the ring 614 engages with the housing as described previously. A pin 619 is then inserted through an aperture of a flange 618 of the ring 614 and through an aperture of a flange 617 of the housing to fixedly couple the ring 614 in position over and around the dial 612.

FIG. 6b illustrates another embodiment 630 in which tampering of the reel system to remove the brace will be evident. Embodiment 630 illustrates a strap 636 that is fixedly coupled to the brace 637 over a guide. The guide is used to initially close the brace during donning of the brace and includes a male component 634 and a female component 632. Additional details of the guide are described in U.S. patent application Ser. No. 14/071,435, titled “Coupling Members for Closure Devices and Systems”, filed Nov. 4, 2013, the entire disclosure of which is incorporated herein by references. Similar to the strap previously described, strap 636 cover the guide to prevent the guide from being uncoupled and the brace from being removed. The strap 626 must be cut to uncouple the male and female components, 634 and 632, and remove the brace. Tampering of the strap 636 would be evident.

FIG. 6c illustrates another embodiment 640 in which tampering of the reel system to remove the brace will be evident. Specifically, the dial 642 includes a plurality of apertures 644 spaced circumferentially around a top portion of the dial 642. After the dial 642 is rotated to tension the lace and close and tension the brace, a wire 647 is inserted through one of the apertures 644 and through a post 646 positioned on the brace or reel system. The wire is crimped by the physician and must be cut to operate the dial 642. Tampering of the reel system would be evident if the wire 647 is cut.

As described briefly above, in some embodiments it may be beneficial and/or desired to allow the patient to adjust the tension of the lace of the closure system within a defined range so that the patient has some control over the fit and/or feel of the brace. For example, the patient may desire to loosen a neck brace when they are relaxing and watching television or may want to tighten the brace when they are engaged in some activity. Such adjustability may provide the patient with some level of comfort, which may help ensure the patient does not get overly annoyed with wearing the brace and will continue to wear the brace.

In some embodiments, the lace tension may be adjusted within a prescribed range to allow the body part to move within a desired range so that an amount of physical therapy may be performed. In other embodiments, the brace may be adjusted within a defined range to compensate for swelling or shrinkage of the body part supported by the brace. Such adjustability may limit the amount of return visits to the physician that are required to compensate for such variability of the body part and/or to perform physical therapy.

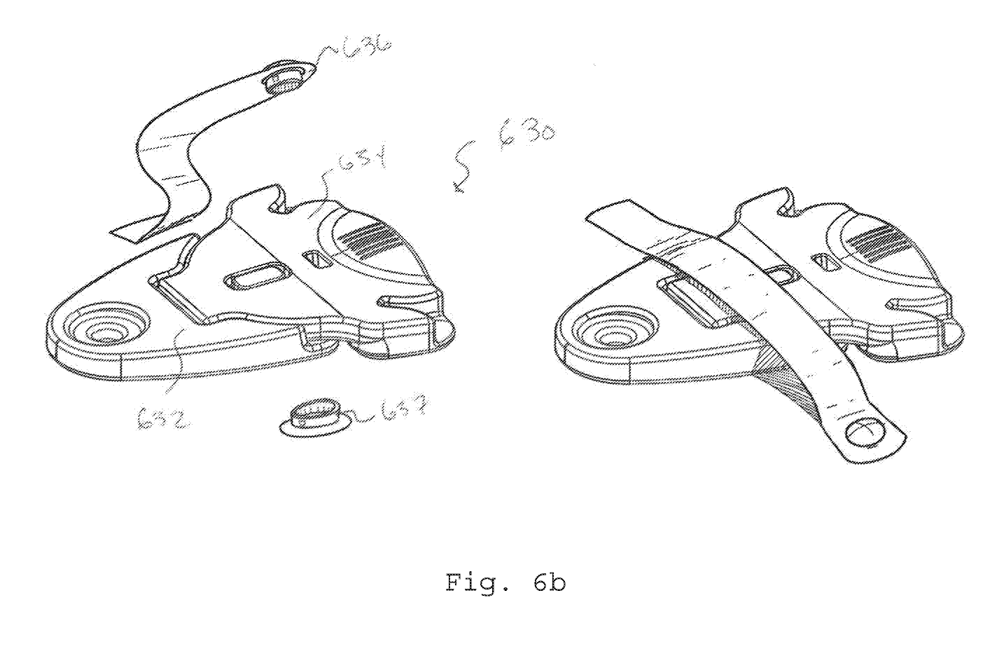

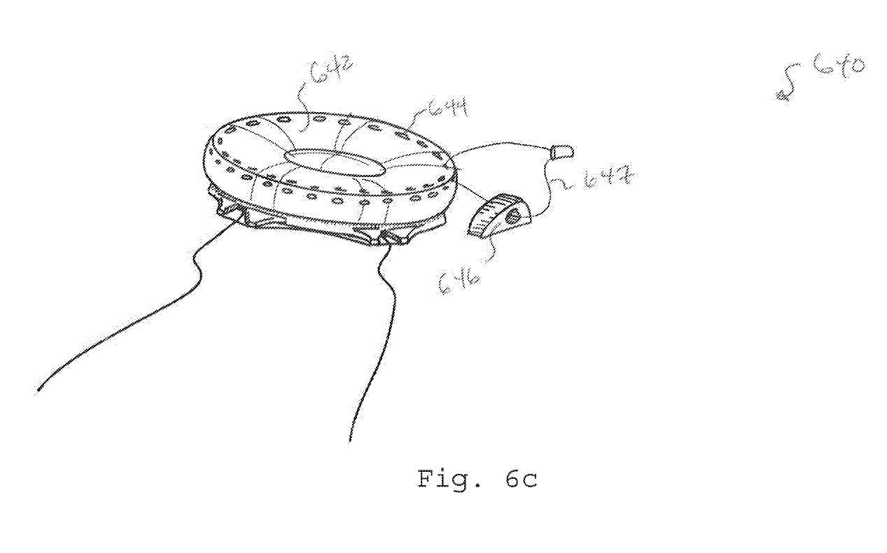

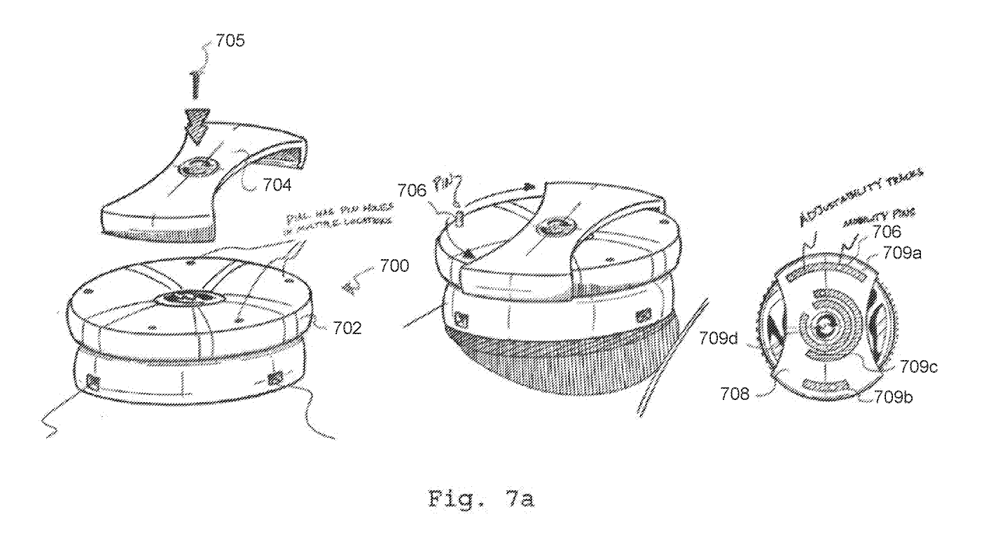

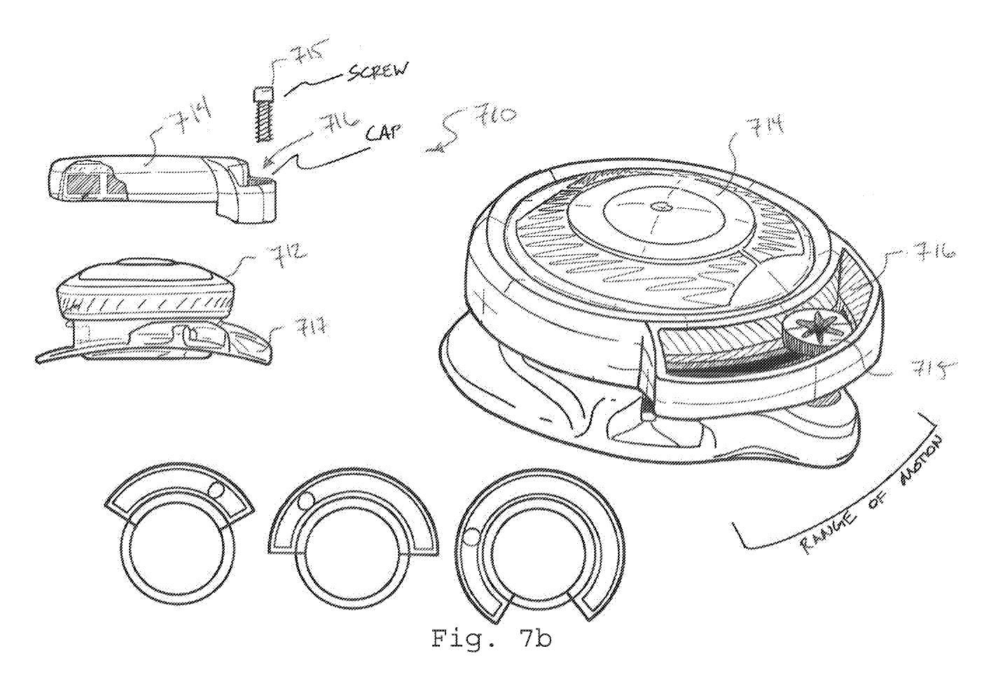

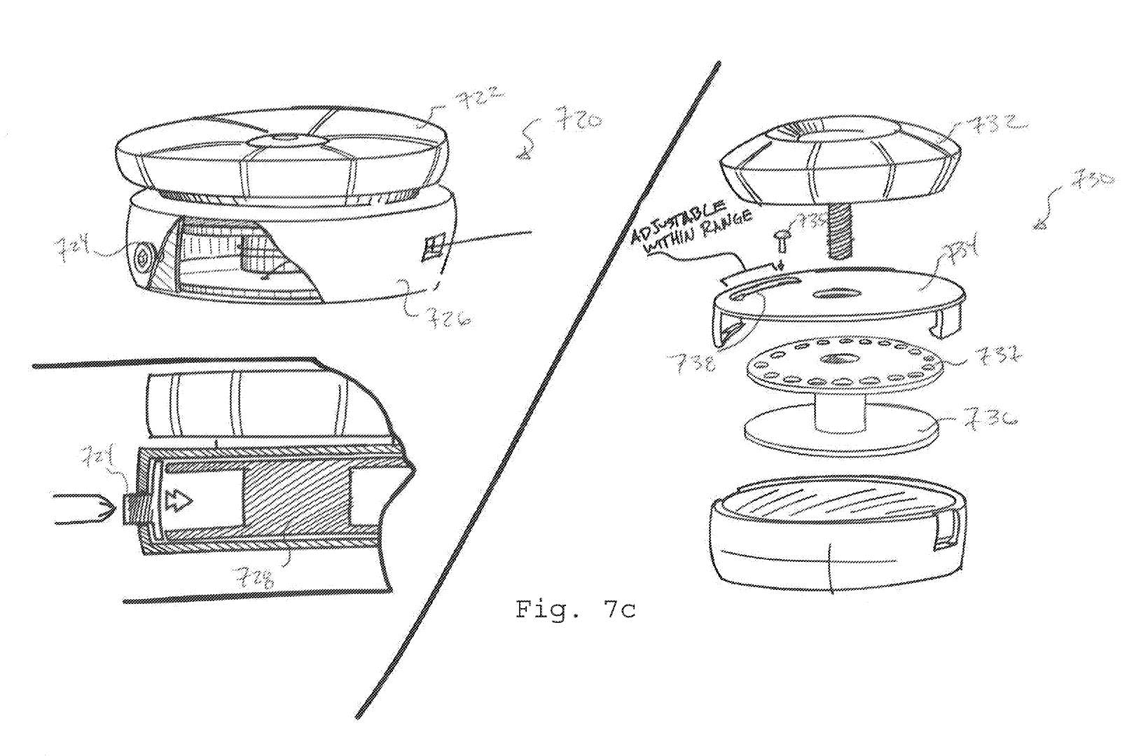

Referring now to FIG. 7a, illustrated is an embodiment of a reel system 700 that allows the user to adjust the lace tension within a defined range. The limited adjustability is provided by fitting a cap 704 atop the dial 702 of reel system 700. The cap 704 may be affixed to the dial 702 by a coupling mechanism 705 that is only removably by the physician. For example, the coupling mechanism 705 may include a screw having a unique head pattern that corresponds to a specially shaped key. The physician may use the specially shaped key to couple the cap 704 with the dial 702.

The dial 702 includes a pin or post 706 affixed to its top surface that contacts the cap 704 as the dial 702 is rotated. The pin 706 prevents the dial 702 from being fully rotated by the patient. The pin 706 allows for some rotation of the dial such that the patient is able to tighten or loosen the lace of the reel system and thus is able to achieve some level of comfort in wearing the brace. As shown on the right hand side of the figure, in some embodiments the pin 706 may be positioned and rotated within a slot 709a. As shown in the right hand figure, the shape of the slot 709a may vary to provide the patient with any desired amount of rotation. The figure shows the slot comprising four configurations 709a-d. In operation, the physician would initially tension the lace via dial 702 and then place cap 704 atop the dial 702, after which the patient would be able to tension the lace with in the defined range by rotating dial 702.

Referring now to FIG. 7b, illustrated is another embodiment of a reel system 710 that may be used to allow a patient to tension a lace within a defined range. Similar to reel system 700, reel system 710 includes a cap 714 that is placed over a dial 712. Cap 714 includes a slot 716 within which a post or screw 715 is positioned. The post or screw 715 is coupled with a housing 717 of the reel system 710. The post or screw 715 rotates within this lot 716 to allow the user to adjust the tension of the lace within a defined range. As shown in the bottom of the figure, the slot 716 may include various shapes to provide any desired range of motion.

FIG. 7c illustrates another embodiment of a reel system 720 that may be used to allow a user to adjust lace tension within a desired range. Specifically, reel system 720 may include a dial 722 that is coupled with a housing 726 as described above. A post or pin 724 may be coupled with the housing 726 such that the post 724 presses against the spool 728. The spool 728 may include a stop member (not shown) that contacts the post 724 as the dial 722 and spool 728 are rotated. Contact between the post 724 and the stop member may limit the amount of rotation of the dial 722. In some embodiments, the spool 728 may comprise an oval or oblong shape such that the spool 728 presses against the post 724 to limit rotation of the dial 722. In another embodiment 730 the reel system may include an additional disc 734 that is positioned between the dial 732 and the spool 736. The additional disc 734 may include a slot 738 within which a pin 735 is positioned. The pin 735 may be inserted within an aperture 737 of the spool and may rotate with in the slot 738 to allow the lace tension to be adjusted within a desired range.

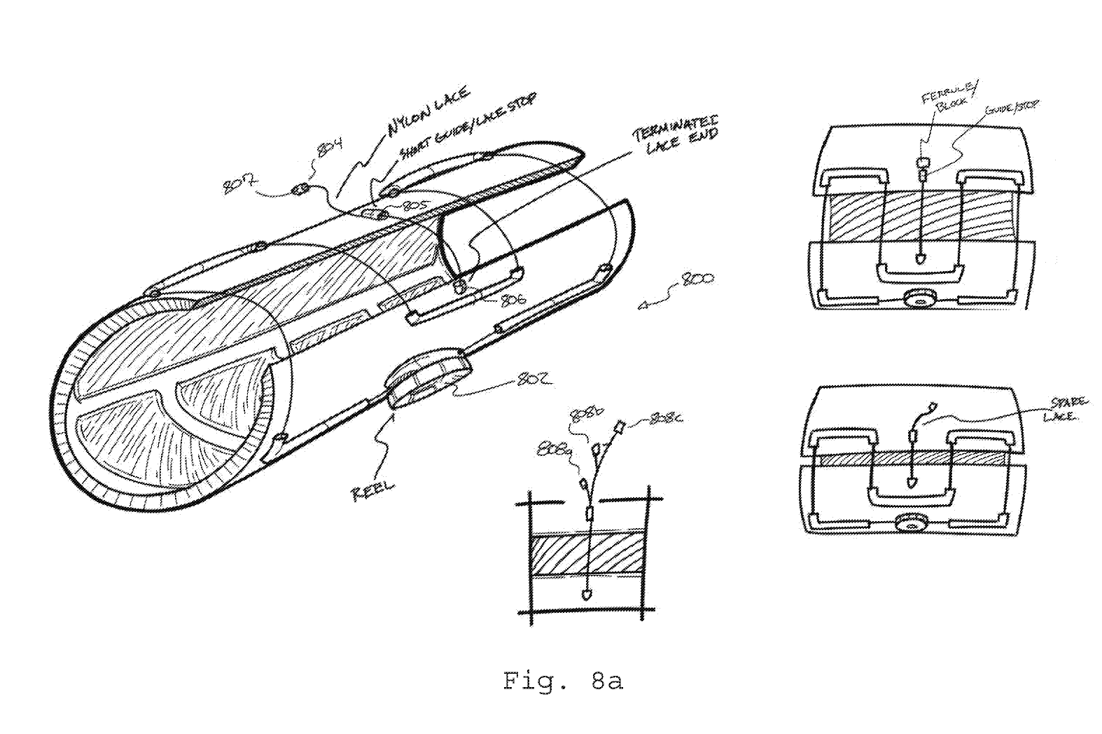

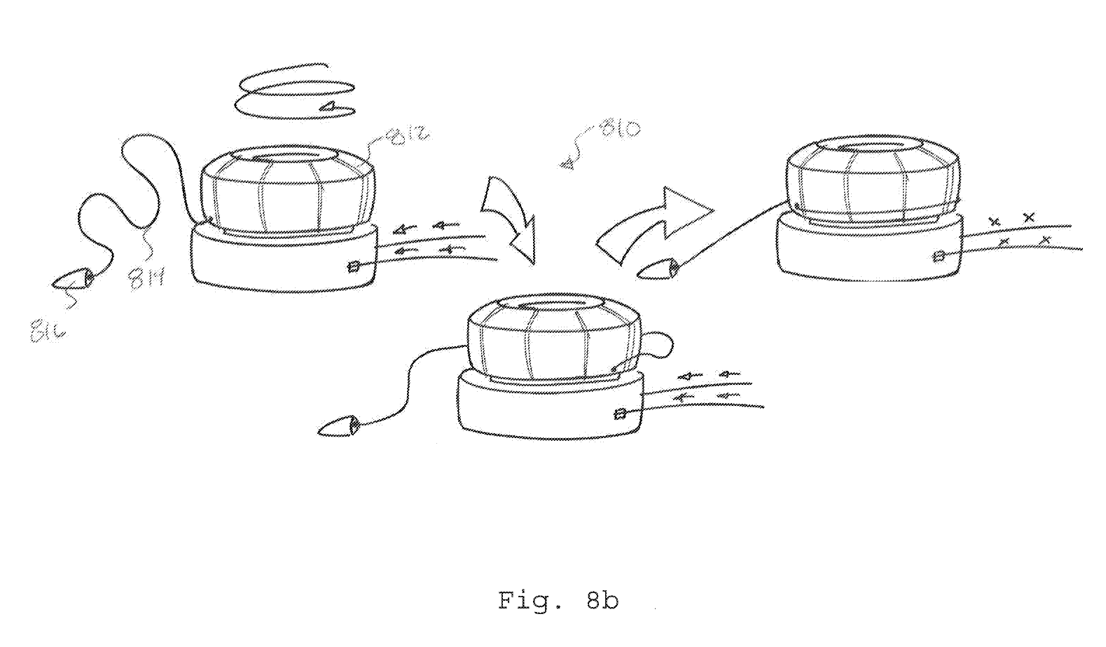

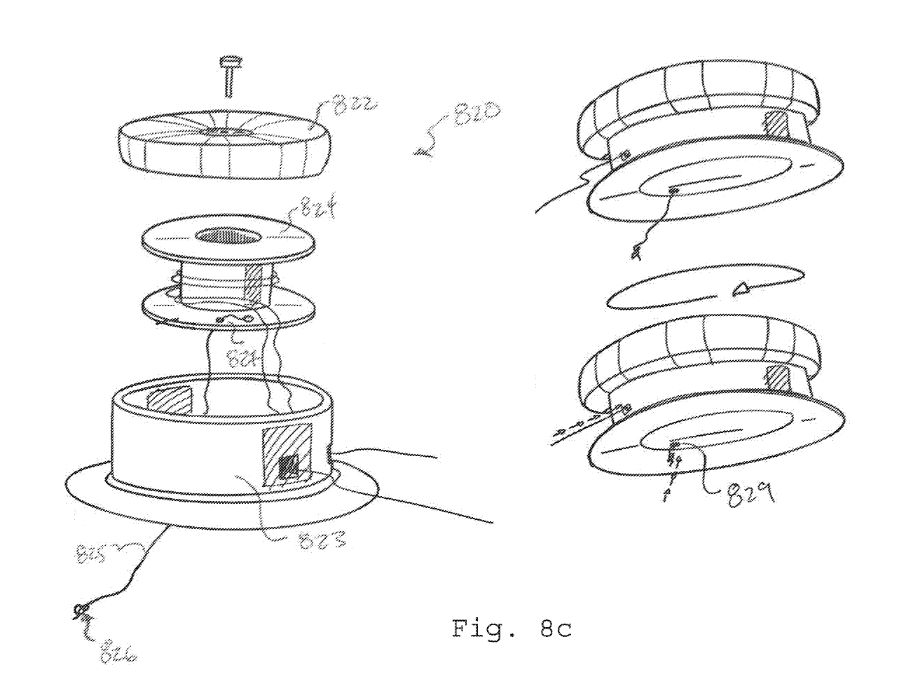

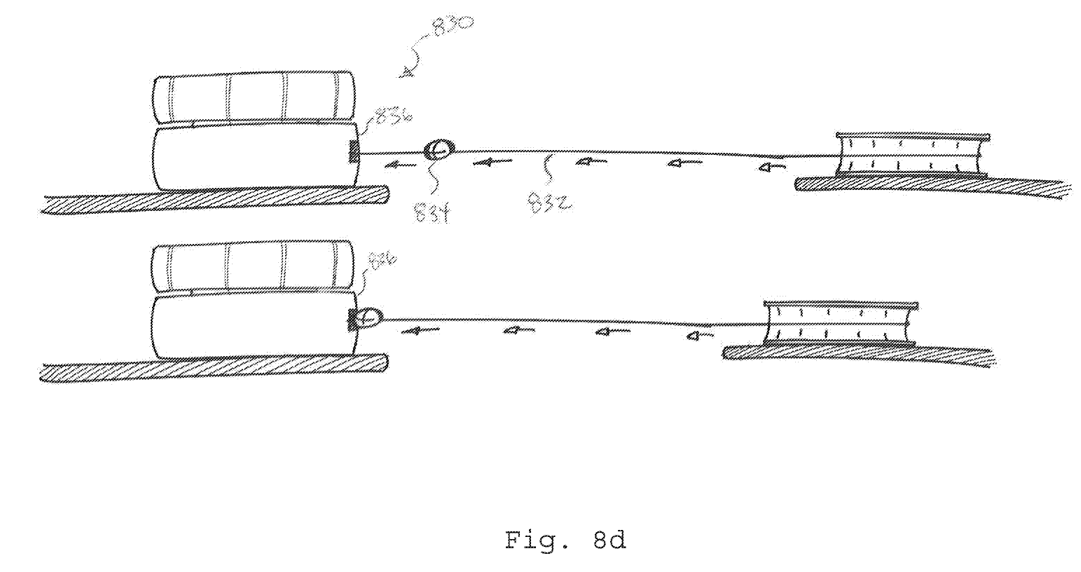

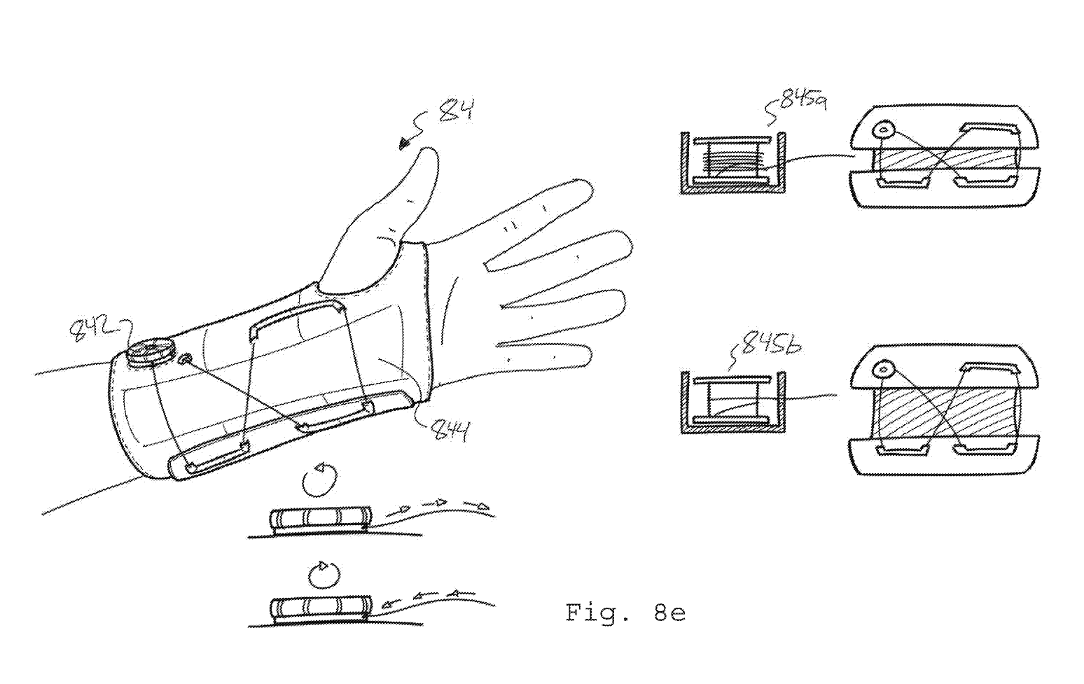

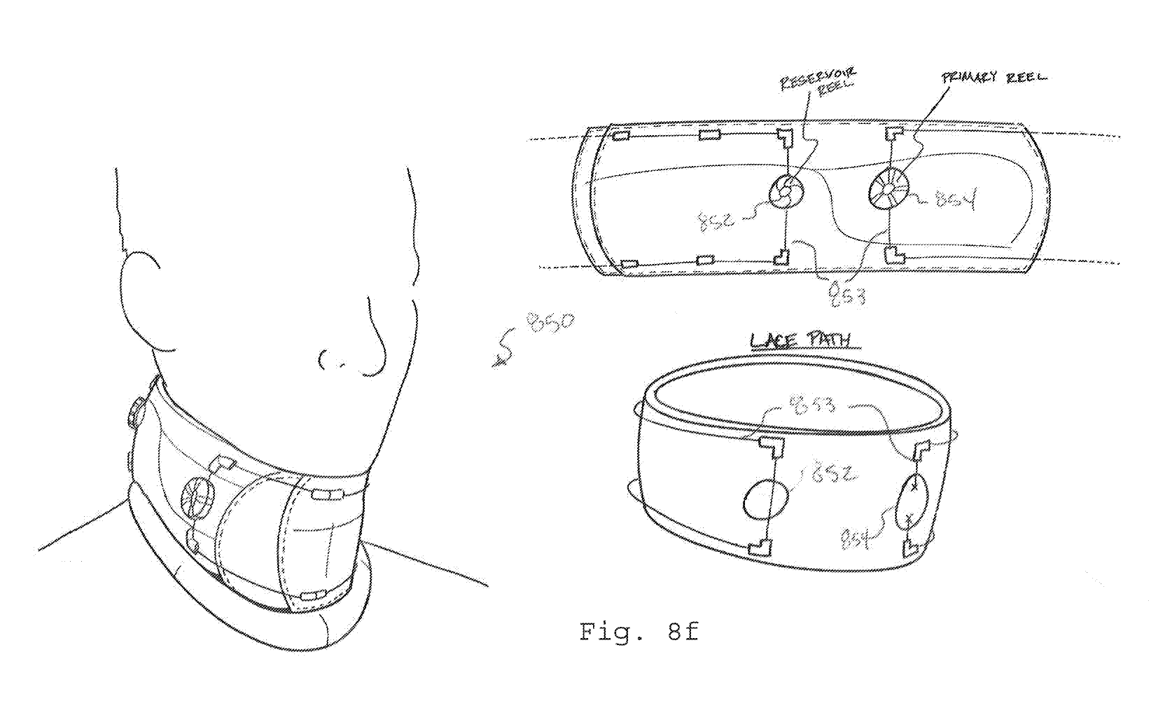

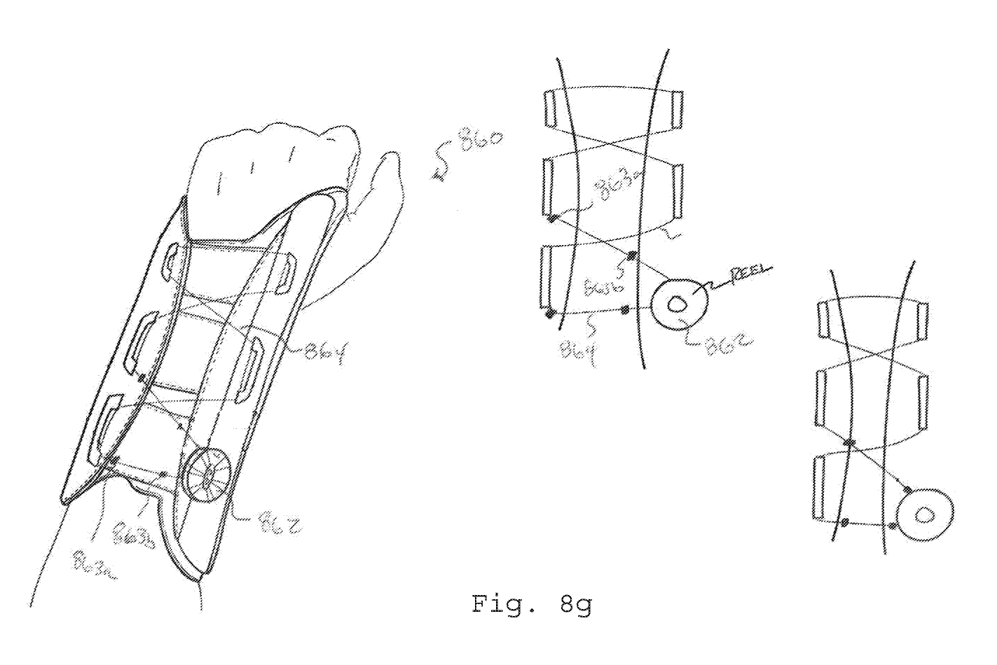

Referring now to FIG. 8a, illustrated is another embodiment 800 of a system for providing lace tension adjustment within a desired range. In this embodiment, a second lace 804 may be used to fix the position of a brace on the patient. The second lace 804 may be coupled with an end 806 that is positioned on one side of the brace and may be slidably inserted through a guide 805 that is positioned on an opposite side of the brace. The second lace 804 may be used to take up slack (i.e., pull out the slack) and then a ferrule or other component 807 may be crimped about the second lace 804 such that the ferrule 807 contacts the guide 805 as the brace is pulled open. Contact between the ferrule 807 and guide 805 may prevent the brace from fully opening. The patient may use the dial 802 to adjust the lace tension of the first lace. As the lace tension is tightened, the second lace 804 and ferrule 807 may slide through and away from the guide 805. As the lace is loosened, the second lace 804 and ferrule 807 may slide through and toward the guide 805 and eventually contact the guide 805 if the lace tension is loosened enough. As shown in the bottom figure, in some embodiments the second lace 804 may include multiple ferrules 808a-c. The multiple ferrules 808a-c may be used by the physician to increase the range of adjustability provided to the patient. For example, the physician could set the brace such that a first ferrule 808a contacts the guide 805 to provide a limited amount of adjustability. After a defined amount of time, such as one week, the physician could clip the first ferrule 808a so that a second ferrule 808b contacts the guide 805 to allow a greater amount of adjustability. After a second defined amount of time, such as an additional week, the physician may clip the second ferrule 808b so that a third ferrule 808c contacts the guide 805 to provide a maximum amount of adjustability. In this manner, the range of adjustability provided to the patient may be varied over a defined amount of time.

Referring now to FIG. 8b, illustrated is another embodiment of a reel system 810 that allows a patient to adjust lace tension within a defined range. Reel system 810 includes a dial 812 that is operated to adjust lace tension as described above. Coupled with one portion of dial 812 is a second lace 814 having an end fixedly coupled with the brace 816 or reel system. As the dial 812 is rotated, the second lace 814 winds around the dial 812 or around a spool inside the dial 812. Because the second lace 812 is fixedly coupled to the brace 816, the second lace 814 limits the amount of rotation of the dial 812. FIG. 8c illustrates a similar concept. The reel system 820 of the FIG. 8c includes a dial 822, a spool 824, and a housing 823 as described above. A second lace 825 is coupled with the spool 824 via an aperture 827 positioned on a bottom flange of spool 824. The second lace 825 exits through an aperture 829 positioned on a bottom surface of the housing 823. A knot 826 is tied at a distal end of the second lace 825 such that the knot 826 is not able to pass through the aperture 829. As the dial 822 is rotated, the spool 824 rotates within the housing 823, which causes the second lace 825 to wind around the spool 824. Winding of the second lace 825 around the spool 824 causes the second lace 825 to be pulled within the housing 823 via an aperture 829. If the spool 824 is sufficiently wound, the knot 826 will contact the aperture 829 to prevent further rotation of the spool 824 and dial 822. The knot 826 may be tied at the end of the second lace 825 after a physician has initially tensioned the lace. The patient may then adjust the latest tension within the range defined by the second lace 825 and the knot 826.

Referring now to FIG. 8d, illustrated is another embodiment of a reel system 830 that may be used to allow a patient to adjust lace tension within a defined range. The reel system includes a dial and housing 836 as described above. The dial is rotated to tension a lace 832. After the lace 832 is initially tensioned by a physician, a ferrule or stop component 834 may be coupled with the lace in a manner such that additional tensioning and/or loosening of the lace 832 will cause the ferrule 834 to contact the housing 836. In some embodiments, the ferrule 834 may be positioned on the lace 832 such that the ferrule 834 contacts a guide (not shown) or other component of the reel system 830 upon tensioning or loosening of the lace 832 via the dial.

Referring now to FIG. 8e, illustrated is another embodiment of a reel system 840 that may be used to provide a patient with a limited range of lace tension adjustability. Reel system 840 includes a dial 842 that is operated to tension a lace 844 as described above. The total lace length of a reel system 840 may be such that the brace may open only by a desired amount. For example, the patient may only be able to loosen the lace 844 until the lace is fully or near fully unwound from the spool 845b. If the lace 844 is wound around the spool only a few times, the brace will not open much as a lace tension is loosened. Similarly, the patient may wind the lace around the spool 845a to close the brace. After a physician initially tensions the lace 844, such as by pulling on the lace end, the lace 844 may be tied to a ring so that the reel system 840 includes only a desired amount of lace 844. The total lace length may be initially set as desired to provide the patient with a desired amount of adjustability.

Referring now to FIG. 8f, illustrated is another embodiment of a reel system 850 that may be used to allow a patient to adjust lace tension within a defined range. The reel system 850 includes a primary reel 854 that is used to initially tension the lace 853 of the reel system 850. The primary reel 854 is then locked so that further lace tensioning or loosening is not permitted. The reel system 850 also includes a reservoir reel 852 that may be used by the patient to further tension and/or loosen the lace 853. In some embodiments, the lace 853 may pass through a lumen or channel of the reservoir reel 852 so that the patient is not able to loosen the lace 853 beyond a lace tension initially set or achieved the primary reel 854. Rather, the reservoir reel 852 may only be usable to tension the lace 853. Stated differently, the patient may only be able to loosen the lace 853 until the lace is aligned with the lumen or channel of the reservoir reel 852, at which point the lace essentially disengaged with the reservoir reel 852 (i.e., at which point the lace is not wound around a spool or annular channel of the reservoir reel). Any subsequent rotation of the reservoir reel 852 in either the clockwise or counterclockwise direction will cause the lace 853 to be tensioned. In other embodiments, a physician may use the reservoir reel 852 to take up a desired amount of slack in the lace 853 and thereby initially tension the lace and close the brace. The reservoir reel 852 may then be locked and the patient may use the primary reel 854 to further tension or loosen the lace 853.

Referring now to FIG. 8g, illustrated is another embodiment of a reel system 860 that may be used to allow a patient to adjust lace tension within a defined range. Reel system 860 includes a dial 862 and lace 864 as described previously. A pair of ferrules 863a and 863b may be positioned on the lace 864 such that tensioning and/or loosening of the lace 864 via the reel 862 causes one of the ferrules to contact a guide or the housing of the reel system 860. For example, if the lace tension is loosened, a first ferrule 863a will contact a guide of reel system 860. Similarly, if the lace tension is tightened a second ferrule 863b will contact the housing of the reel system 860. In some embodiments, a pair of first and second ferrules may be positioned on the lace 864 as shown.

Referring now to FIG. 9a, illustrated is another embodiment of a reel system 900 that may be used to allow a patient to adjust placed tension within the desired range. Reel system 900 includes a dial 902 that is used to tension lace 903 as described herein. After the lace 903 has been initially tensioned, an adjustable guide, such as a turnbuckle 904, positioned at a distal end of the lace 903 may be used to further tension or loosen the lace 903. The turnbuckle 904 is connected with threaded rods 908 that are in turn threadingly coupled with a first and second ends, 906a and 906b. As the turnbuckle 904 is rotated, the threaded rods 908 thread into or out of the first and second ends 906a and 906b. The dial 902 may be locked as described herein to prevent further tensioning and/or loosening of the lace 903. Accordingly, the patient may only be able to tension or loosen the lace 903 via the turnbuckle 904. The threaded rods 908 may be coupled with the first and second ends 906a and 906b to prevent the threaded rods 908 from being fully withdrawn from the first and second ends. Further, the threaded rods 908 and the first and second ends 906a and 906b may be coupled to provide a desired amount of lace tension adjustability.

FIG. 9b illustrates another embodiment of a reel system 910 that may be used to allow a patient to adjust lace tension within a defined range. Reel system 910 includes a dial system 912 and lace as described above. A coupling buckle 916 may be coupled with the lace of reel system 910 and further coupled with webbing 919 that is connected to the brace, such as via a male releasing guide 914. The reel system 910 further includes a webbing take-up mechanism 918 that is coupled with the webbing 919. The take-up mechanism 918 is configured to release the webbing 918 after the lace is tensioned beyond a certain amount and/or to take-up (i.e., wind) the webbing 919 after the lace is loosened beyond a certain amount. In this manner, the tension on the lace may be maintained within a desired range.

Referring now to FIG. 10a, illustrated is another embodiment of a reel system 1000 that may be used to allow lace tension adjustability within a defined range. The reel system 1000 includes reel components as described herein. Specifically, the reel system 1000 includes a core 1004, a pawl ring or disc 1002, a spool 1006, a housing 1008, and a dial (not shown). A post or stop block 1007 is coupled with a bottom surface of the spool 1006. The post 1007 is inserted within a channel or track 1009 positioned on the bottom surface of the housing 1008. The post 1007 slides within the track 1009 as the spool 1006 is rotated via the dial. Rotation of the spool 1006 is limited as the post 1007 contacts edges of the track 1009. In an alternate embodiment, the post 1007 may extend radially from the spool and slide within a track positioned on circumferentially around an interior wall of the spool 1006. After initially tensioning the system, the post 1007 could be inserted from outside of the housing 1008 into an available spool hole.

In one embodiment, the physician may use a first spool (not shown) to initially tension the lace of reel system 1000. In one embodiment, a primary reel (not shown) may be used to initially tension the lace and the reel system 1000 may be used as the reservoir reel previously described. In other embodiments, any of the means of initially tensioning the lace may be used, after which the reel system 1000 may be used to additionally tension and/or loosen the lace as described herein. In another embodiment, the reel may be tensioned and a post then inserted through the bottom of the housing. The reel may then be snapped into a bayonet.

FIG. 10b illustrates another reel system 1010 that may be used to allow lace tension adjustment within a defined range. The reel system 1010 includes a dial 1012 that is used to tension the lace as described herein. The reel system 1010 further includes a cartridge 1014 that may be coupled with the brace and operationally coupled with the dial 1012 to limit the rotational movement of the dial 1012 and, thus, the ability of the dial 1012 to further tension/loosen the lace. The cartridge 1014 includes a gear that couples with a gear of dial 1012. Coupled atop the gear of the cartridge 1014 is a finger 1016 that rotates and contacts a stop member 1015 to prevent additional rotation of the cartridge's gear and further rotation of the dial 1012. The cartridge 1014 may be a separate unit that is snap fit to the brace and the dial 1012 after the lace is initially tensioned by the physician. Further, the cartridge used may be selected to provide a desired gear ratio between the dial 1012 and the cartridge that allows a desired amount of dial rotation and, thus, provides a desired amount of patient lace tension adjustability. In some embodiments, the lace may be coupled with the cartridge so that the lace is initially tensioned via the cartridge and the cartridge is coupled with the brace to allow further tensioning of the lace via the dial 1012.

In another embodiment, a single dial system 1030 may be used instead of using a cartridge. In the single dial system 1030, the finger 1033 and stop member 1037 may be components that are positioned within the dial system 1030. For example, the finger 1033 may be coupled with a disc 1034 that is positioned between the dial 1032 and spool 1036. As the dial 1032 and spool 1036 rotate, the finger 1033 of the disc 1034 may contact the stop member 1037 that extends radially inward from the housing 1038.

FIG. 10c illustrates another embodiment of a reel system 1020 that may be used to provide lace tension adjustability within a desired range. Specifically, the reel system 1020 includes a stop component 1021 that provides a desired amount of rotation of the dial 1027. The stop component 1021 includes a plurality of fingers 1023a-n that each have an axially extending post. The axially extending posts contact an adjacent axially positioned finger as the finger rotates around a central axis of the stop component 1021. For example, as a first finger 1023a rotates, the finger eventually contacts the axially extending post of the finger 1023n positioned axially below the first finger. The two fingers (i.e., the first finger 1023a and the finger 1023n positioned axially below the first finger) then rotate together until an axially extending post of another finger 1023n+1 is contacted. Rotation of any contacted fingers continues until a post of a bottom finger that is fixedly coupled to a bottom flange 1024 of the stop component 1021 is contacted, which prevents further rotation. Rotation in the opposite direction works similarly, although in reverse. The first finger 1023a is fixedly coupled to a top flange 1022 of the stop component 1021, such as by inserting the axially extending post within an aperture of the top flange, so that the first finger 1023a rotates with the top flange 1022.

The stop component 1021 provides roughly N−1 revolutions of the dial 1027, where N represents the number of fingers 1023a-n used in the stop component. For example, if the stop component includes 4 fingers, the component allows the dial 1027, and thus spool 1026, to rotate slightly less than 3 full revolutions. The stop component 1021 may be positioned within the reel system 1020, such as between the spool 1026 and housing 1028. The stop component 1021 may include teeth on the top flange 1022 that contact corresponding teeth (not shown) of the spool 1026.

Referring now to FIG. 10d, illustrated is another embodiment of a reel system 1040 that may be used to provide limited lace tension adjustability. The reel system 1040 includes two gears that are used to limit the rotation of a knob 1043 of the reel system 1040. Specifically, the reel system 1040 includes a spool 1042 that is similar to the spools of the other reel systems described herein except that spool 1042 includes a finger 1045 that is used to limit the rotation of knob 1043. Rotation is limited via the use of an idler gear 1044 that also includes a finger 1046. As the spool 1042 is rotated via the knob 1043, the idler gear 1044 and fingers, 1045 and 1046, also rotate. The spool 1042 may be rotated until the fingers, 1045 and 1046, contact one another, which prevents further rotation. The fingers, 1045 and 1046, may contact each other in an initial position and final position to limit rotation of the spool 1042 in both a forward and reverse direction. The arrangement of the fingers, 1045 and 1046, and/or size of spool 1042 and idler gear 1044 may be configured so as to provide a desired amount of rotation in both the forward and reverse directions.

Referring now to FIG. 10e, illustrated is another embodiment of a reel system 1050 that may be used to provide limited lace tension adjustability. Reel system 1050 includes a threaded drive shaft 1054 that is coupled with the spool 1052 of reel system 1050. The drive shaft 1054 rotates as the knob 1053 and spool 1052 are rotated. A block 1056 is slidably coupled with the drive shaft 1054 so as to move axially along the drive shaft 1054 (via threads) between a first position 1058a and a second position 1058b as the knob 1053 and spool 1052 are rotated. The block 1056 may also be slidably coupled with a second shaft 1057 that stabilizes the block 1056 as it slides along the drive shaft 1054. The block 1056 is configured to contact a wall at the first position 1058a and the second position 1058b to limit further axial movement of the block 1056 and thus, limit further rotation of the knob 1053 and spool 1052. In this manner, the lace tension adjustability is limited. The drive shaft 1054 and block 1056 may be configured to provide a desired amount of lace tension adjustability.

As described above, in some embodiments it may be desirable to fixedly attach guides of a reel system about the brace so as to “lock” the brace about the body part. Stated differently, it may be desirable to close the brace about the body part via lace guides of the reel system and then fixedly couple the lace guides so that the patient is not able to remove the brace. After fixedly coupling the lace guides, the patient may be able to further tension and/or loosen the lace via the limited adjustability features described herein so as to achieve an amount of comfort in wearing the brace.

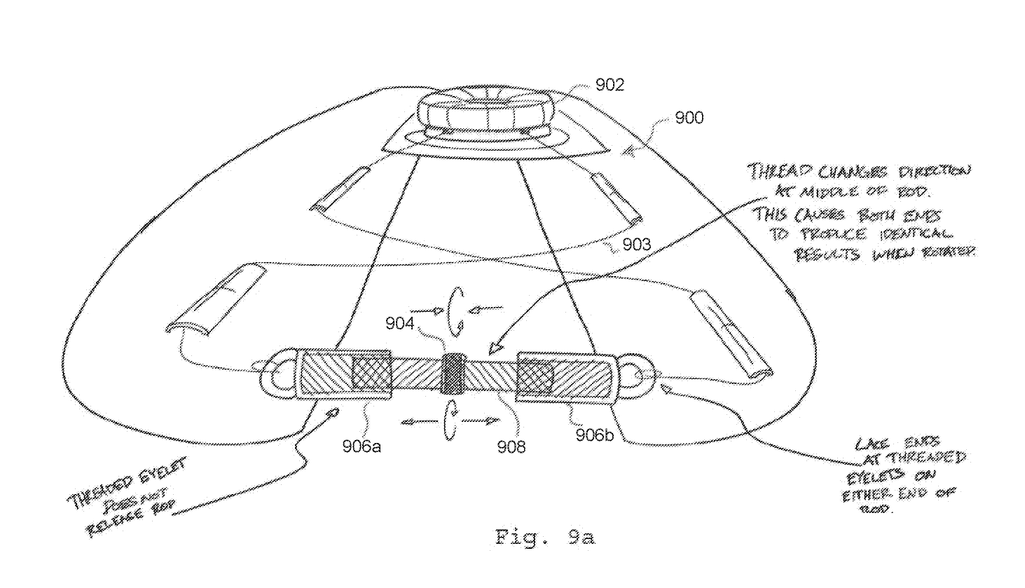

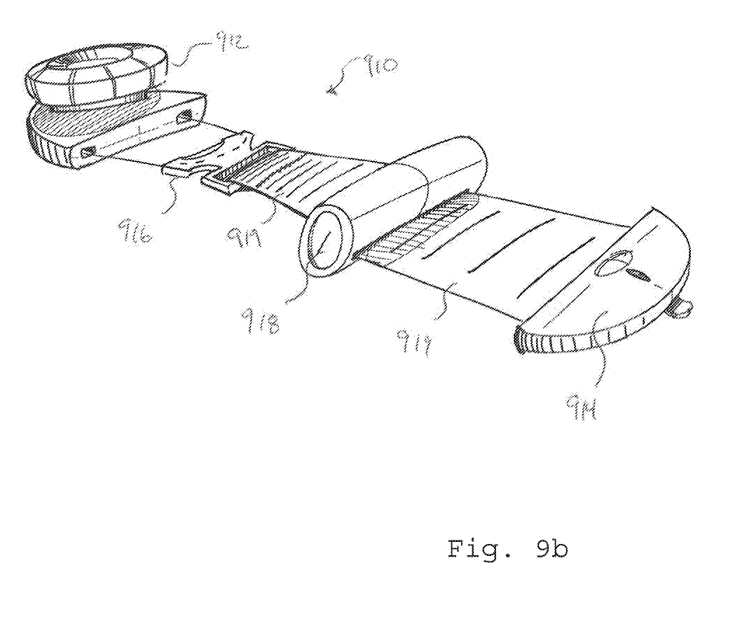

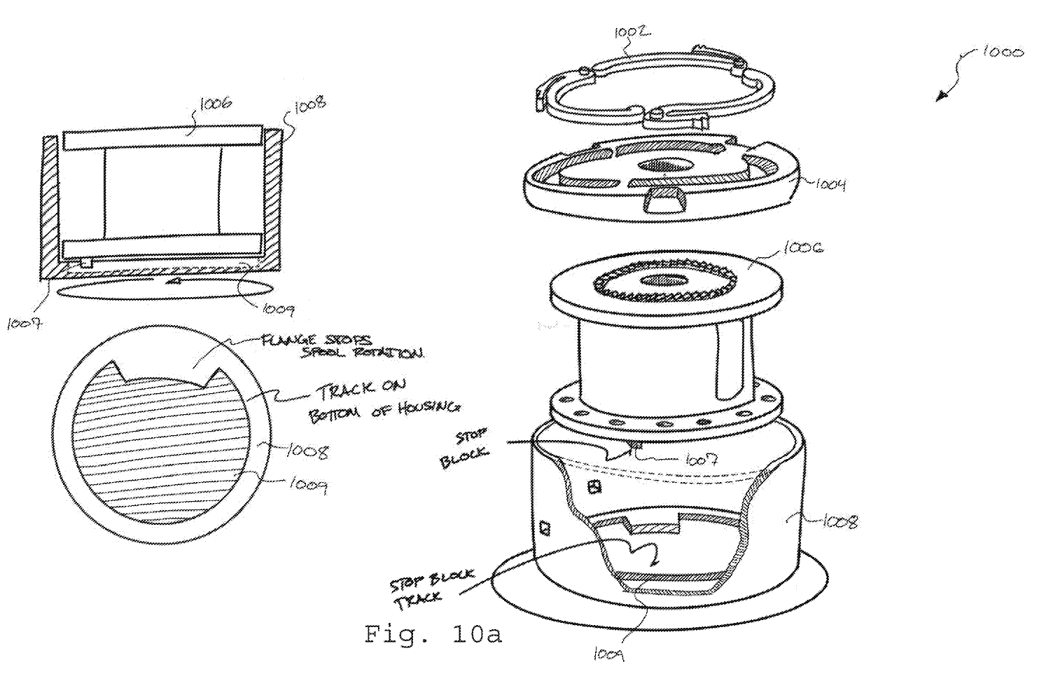

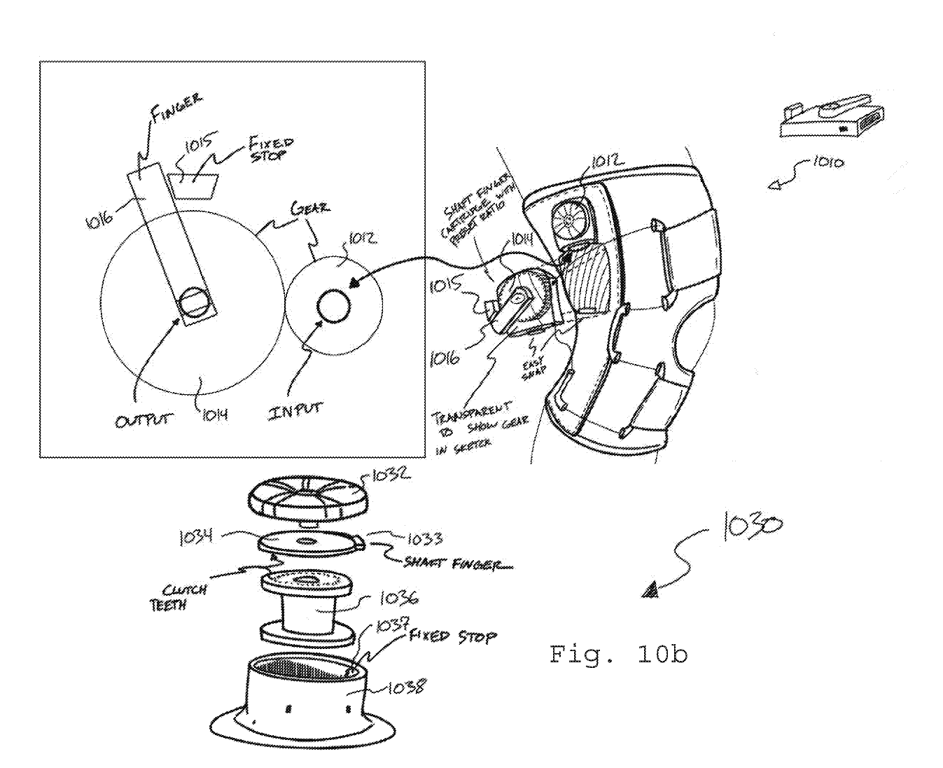

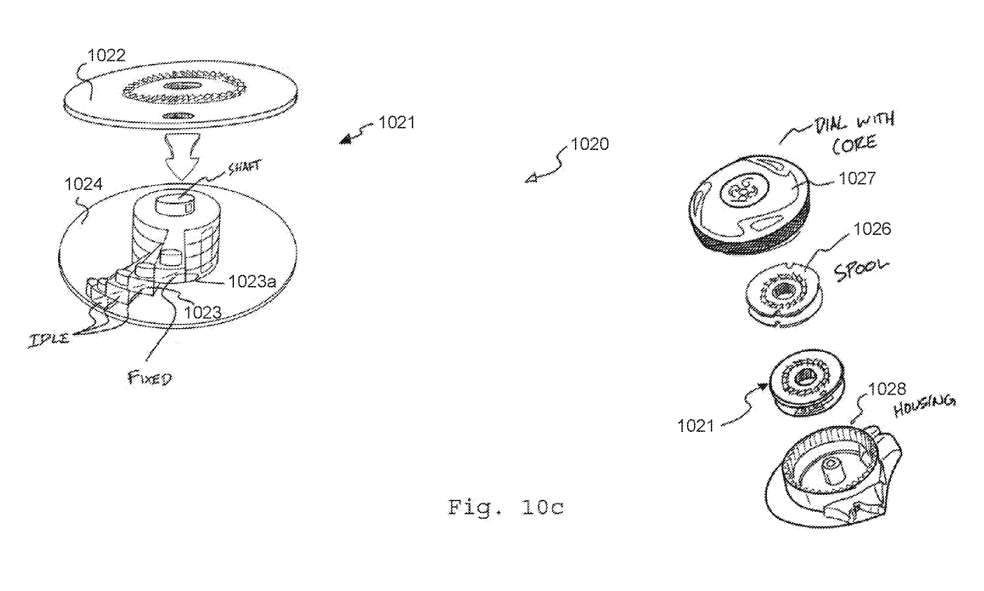

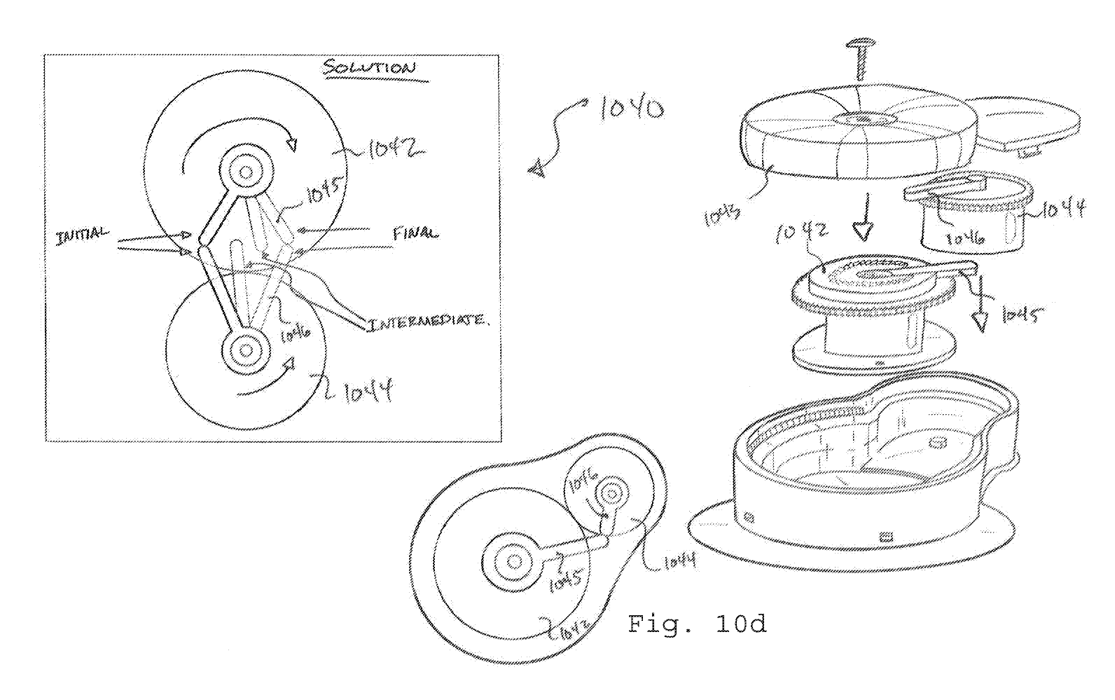

Referring now to FIG. 11a, illustrated is one embodiment of a lace guide system 1100 that may be fixedly coupled to the brace to “lock” the brace about the patient's body part. Lace guide system 1100 includes a female component 1102 and male component 1104 as described herein and as further described in U.S. patent application Ser. No. 14/071,435 incorporated by reference herein. Female component 1102 includes a post 1107 that fits within a corresponding slot 1105 of the male component 1104. A coupling mechanism, such as a lockable pin or screw 1106, is inserted through the slot 1105 and into post 1107. In some embodiments, the pin or screw 1106 may be press fit into an aperture of the post 1107. In other embodiment, the pin or screw 1106 may be threaded into the post 1107. The pin or screw 1106 may include a specially keyed configuration that allows the pin or screw 1106 to only be removed with a special corresponding tool.

FIG. 11b illustrates other embodiments of lace guide systems that may be used to lock the brace about the patient's body party. Specifically, the embodiment 1110 shows a male component 1114 including an angle member 1116 that is insertable within a chamber 1115 of female component 1112. The angled member 1116 is made of a resilient material to allow the angled member 1116 to deflect angularly closed as the angled component 1116 is inserted within the chamber 1115. The angled component is also biased toward an angularly open position which causes an edge of the angled component 1116 to contact an inner edge of the chamber 1115 as the male component 1114 is pulled proximally relative to the female component 1112. The female component 1112 may include an aperture 1117 that allows a special tool to be inserted within the chamber 1115 to press the angled component 1116 toward an angularly closed position, which disengages the edge of the angled component 1116 from the inner edge of the chamber 1115 to allow the angled component 1116 to be withdrawn from the chamber 1115.

In another embodiment 1120, a cap 1126 may be fixedly coupled over the coupled male and female components, 1124 and 1122, to prevent uncoupling of the components by the patient. In yet another embodiment 1130, a pin 1136 may be inserted through an aperture 1134 of the female component 1132 after the male and female components, 1134 and 1132, are coupled together. The pin 1136 may fixedly couple the male and female components, 1134 and 1132, such as being inserted through an aperture (not shown) of a stepped flange (not shown) of the male component 1132. The stepped flange is more fully described in U.S. patent application Ser. No. 14/071,435.

FIG. 11c illustrates another lace guide system 1140 that may be fixedly coupled with the brace to lock the brace about a patient's body part. In this embodiment, an aperture 1142 of the male component 1148 is inserted over a post 1144 of the female component 1146 as the components are mated together. The lace of the reel system is then tensioned and the brace closed so that the lace continually tensions the male component 1148. The system may be configured according to any of the embodiments described herein, or in any other manner, so that tension is continually provided to the male component 1148. The continual tension placed on male component 1148 does not allow the male component to be rotated sufficiently relative to the female component 1146 to allow the post 1144 to be removed from the aperture 1142. Accordingly, the male and female components, 1148 and 1146, remain fixedly coupled.

FIG. 11d illustrates an embodiment of a lockable guide 1150 that may be used to ensure that a patient complies with a physicians recommended usage of a brace. The guide 1150 includes a body 1152 that may be coupled with a brace (not shown) via stitching, adhesion, rf welding, mechanically fastening, and the like. Body 1152 includes a channel 1151 within which the lace 1156 is inserted, similar to many of the guides described herein. The body 1152 also includes a centrally located protrusion 1154 that defines a second channel 1155 around which the lace 1156 may be positioned. The second channel 1155 is generally open so that the lace 1156 may be pulled over and around the protrusion 1154. This configuration allows a physician to easily adjust the tension and/or fit of the lace 1156 about a body part. For example, the physician can fit the brace over the patient's body part and pull the lace 1156 into position within second channel 1155 to close the brace about the patient's body part.

When the lace 1156 is within the second channel 1155, a cap 1158 may be pulled or positioned over second channel 1155 and coupled with protrusion 1154 (via mechanism 1159) to lock the lace 1156 within the second channel 1155. A reel assembly or system, such as those described herein, may be then used to finely adjust the tension of lace 1156 and/or the fit of the brace about the body part. In this manner, the patient may be unable to remove the brace from the body part until a treatment plan or therapy regime is completed. In some embodiments, the mechanism 1158 may be a lockable snap, screw, pin, post, adhesive bond, rf weld, and the like.

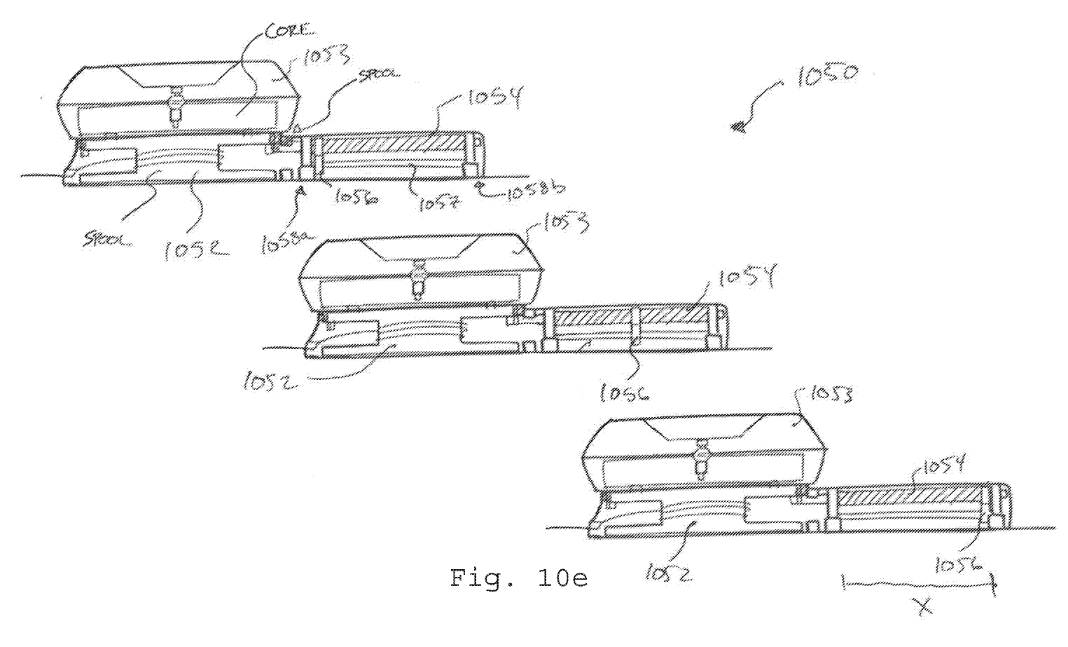

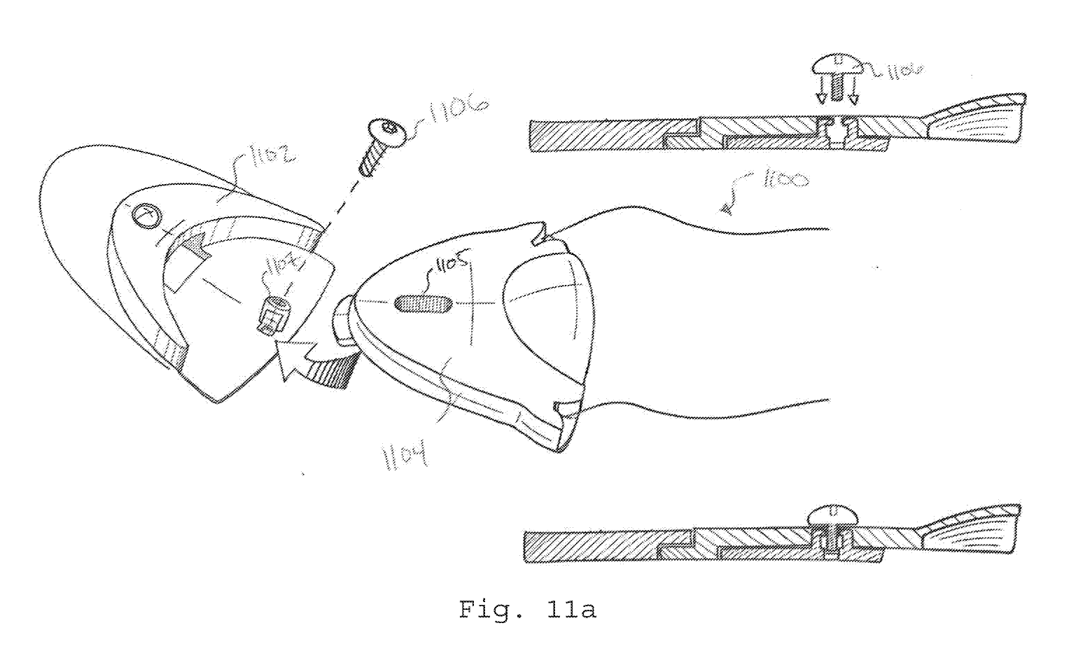

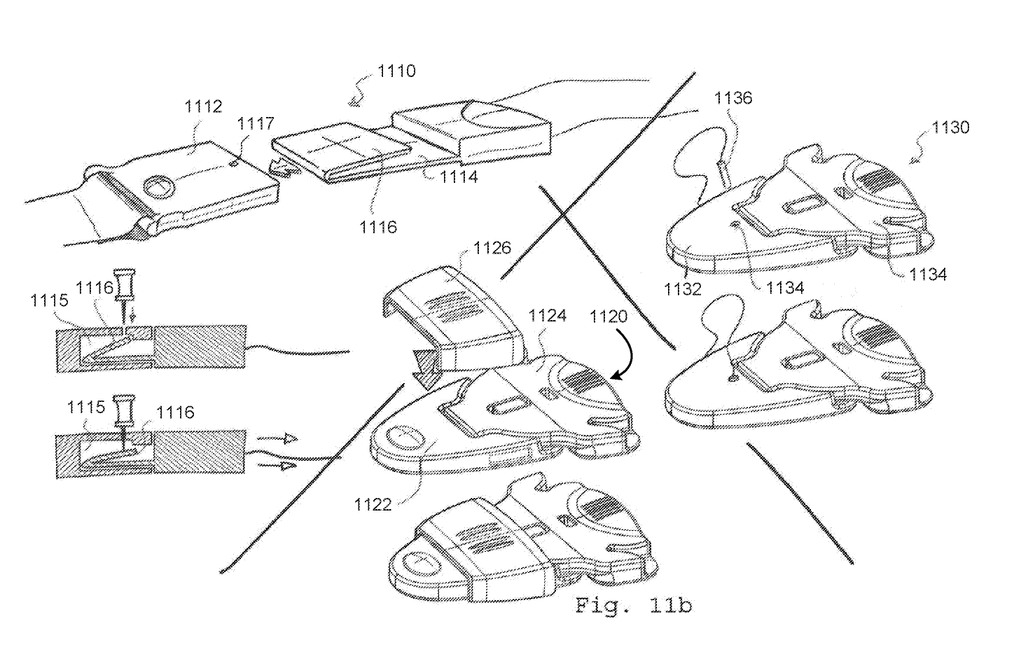

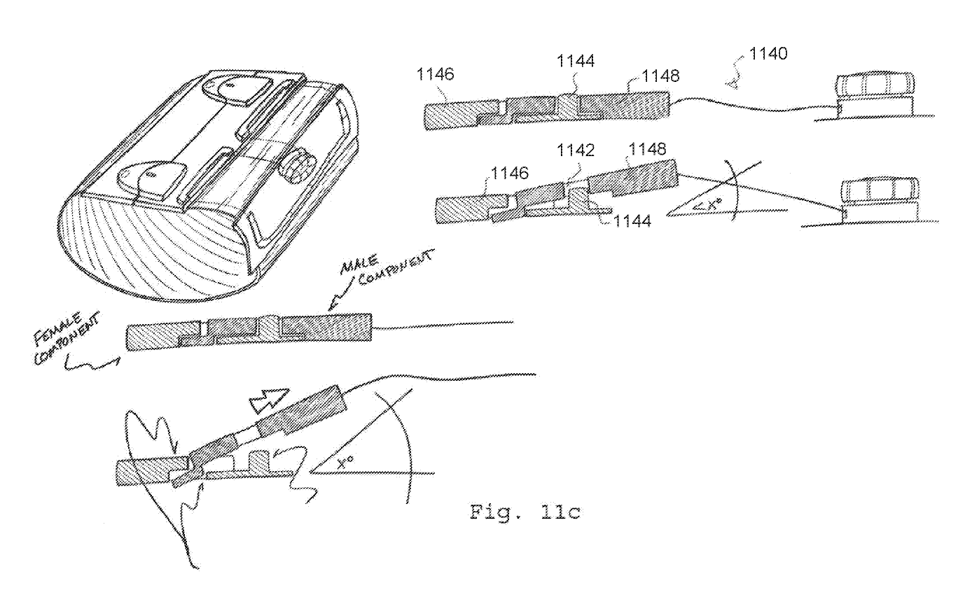

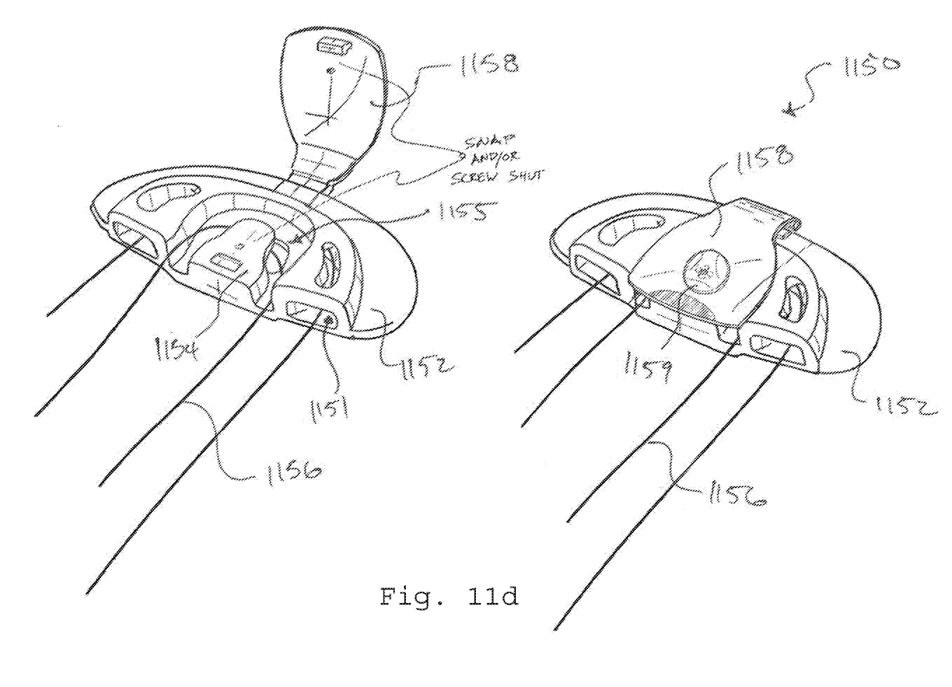

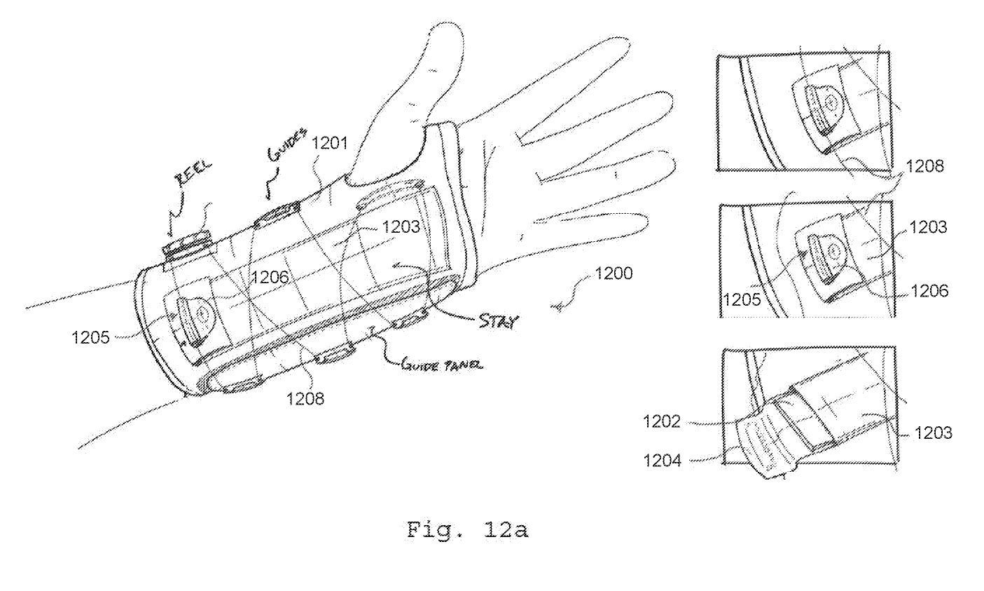

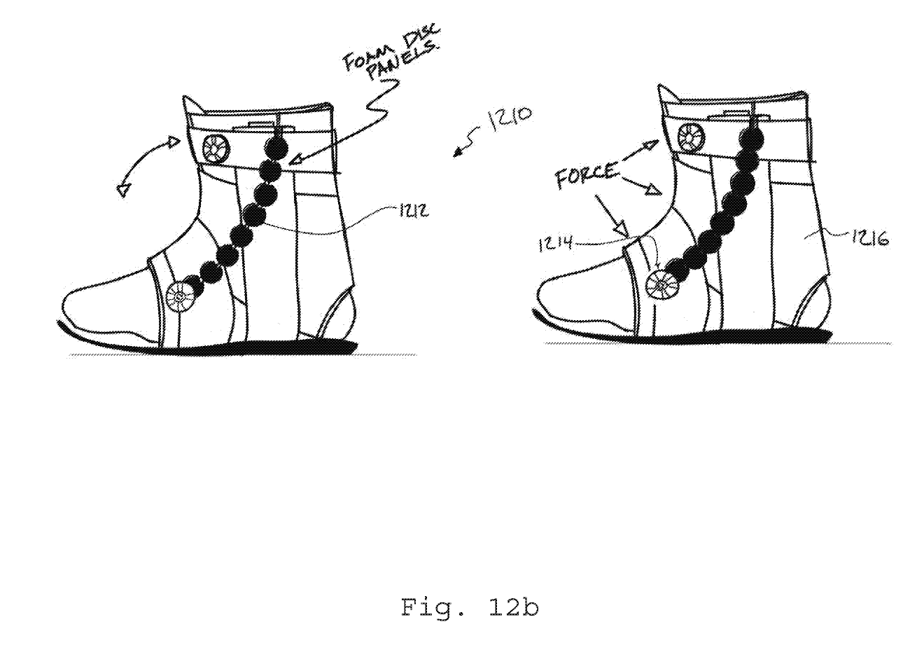

Referring now to FIG. 12a, illustrated is an embodiment 1200 of a brace 1201 having a removable stay 1202. As described herein, a stay 1202 may be a piece of material or component that is used with a brace 1201 to provide a desired brace stiffness. It may be desirable to remove the stay 1202 and replace it with another stay to vary the stiffness of the brace 1201. This may be desirable to vary the flexibility of the brace 1201 so as to allow for increased range of motion and/or to provide physical therapy. For example, the brace 1201 may be initially fit with a relatively stiff stay 1202 to limit the motion of the patient's body part. The stiff stay 1202 may then be removed and replaced with a more flexible stay to provide a limited range of motion and thereby allow the patient to flex the body part and prevent muscle atrophy. Increasingly flexible stays may subsequently be used to provide additional range of motion.

In one embodiment, the stay 1202 may be removable from the brace 1201 by inserting the stay 1202 within a sleeve 1203 and covering an opening of the sleeve 1208 with a flap 1204. The flap 1204 may be coupled with a guide 1206 having a channel 1205 within which lace 1208 of the reel system is placed. As the lace 1208 is tensioned via dial 1207, the lace 1208 presses against the channel 1205 of guide 1206 so as to keep the flap 1204 closed over the opening of sleeve 1203. The dial 1207 may be locked or adjustable within a range as described herein to prevent the patient from loosening the lace to a point at which the lace may be removed from channel 1205 and stay 1202 removed from sleeve 1203. A physician could subsequently loosen the lace 1208 via the dial 1207 to remove the lace 1208 from the channel 1205 and remove and replace the stay 1202 from the sleeve 1203.

FIG. 12c illustrates a similar concept in which the flexibility of a brace may be adjusted. The embodiment 1220 of FIG. 12c illustrates a plurality of panels 1222 that may be slid atop one another to adjust the section modulus of a brace 1226 and thereby vary the stiffness of the brace. A dial system 1224 may be operated to tension the lace and move the panels 1222 atop one another. The dial system 1224 may be locked or adjustable within a range as described herein to allow a physician to set and adjust a stiffness of the brace 1226 as desired. Similarly, the patient may be provided with some range of adjustability of the brace's stiffness to achieve an optimal level of comfort and/or desired fit as described herein. FIG. 12b illustrates a similar embodiment 1210 in which discs 1212 are pulled together via a reel system 1214 to increase a stiffness of a brace 1216. The discs 121 may interlock when pulled together to increase the stiffness of the brace 1216. The physician could set and adjust the stiffness of the lace and/or the patient could adjust the stiffness within a range using the lockable or adjustable within a range concepts described herein.

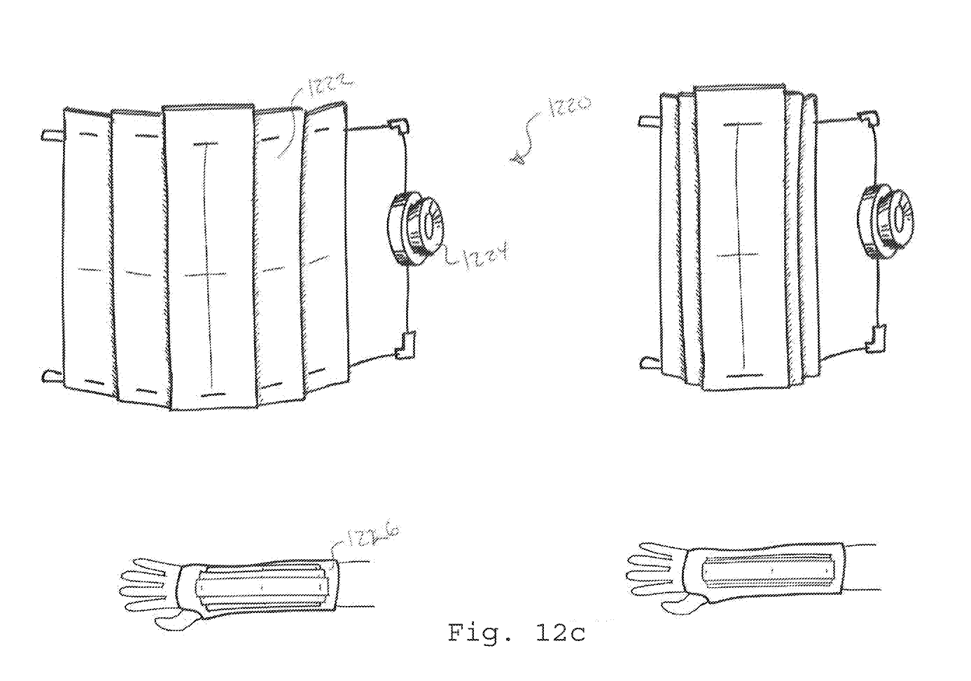

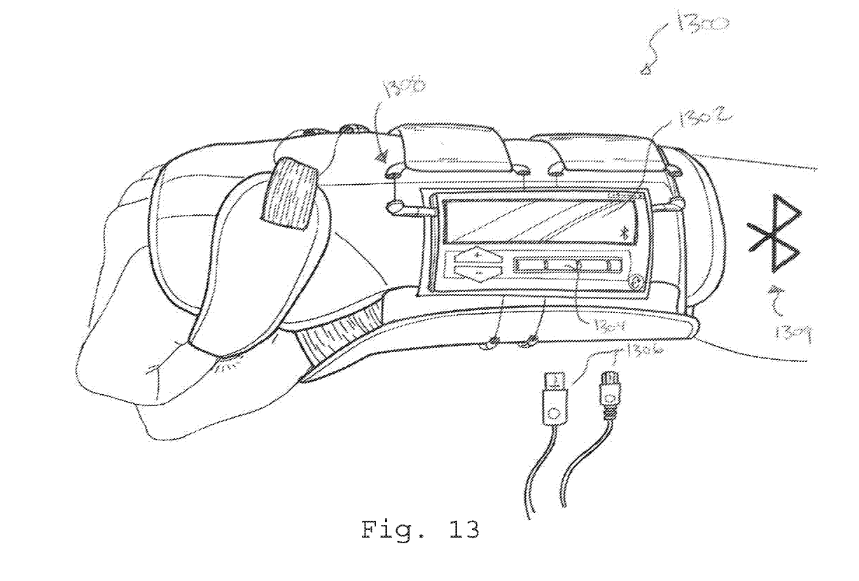

FIG. 13 illustrates an embodiment of a motorized closure device 1300 that may be used to set and or adjust a lace tension of a brace. The motorized closure device 1300 includes a motorized reel system (not shown) that automatically tensions lace 1308 of the reel system. The motorized closure device 1300 includes a display screen 1302 that displays various information to a user, such as lace tension, an amount of time the brace has been worn, a treatment plan for the brace, and/or various other information. The motorized closure device 1300 also includes various input buttons 1304 that allow a physician and/or the patient to set and/or adjust one or more parameters of the system. For example, the lace tension may be set and/or adjusted via the input buttons 1304. A treatment plan could likewise by input into the system, such as an initial lace tension and subsequent lace tensions over a defined amount of time that provide the patient with increased body part flexibility.

In some embodiments, the motorized closure device 1300 may be coupled with an external computing device via one or more cords 1306 that are inserted into ports (not shown) of the motorized closure device 1300. In this manner, information (i.e., tension limits, treatment plans, user compliance reports, and the like) may be communicated (i.e., uploaded and/or downloaded) with the motorized closure device 1300. In some embodiments, the motorized closure device 1300 may communicate and receive information wirelessly 1309 from external computing devices. This may allow a physician to remotely monitor a patient's compliance with a treatment plan and/or adjust the treatment plan as needed. In other embodiments, the physician may transmit messages to the patient that are displayed on the display screen 1302, such as reminders to perform any needed physical therapy.

In some embodiments, the motorized closure device 1300 can be programmed to allow the patient to adjust the lace tension within a desired range and/or allow the patient to remove the brace for a prescribed period of time. Reminder messages may be sent to the patient if the brace is not refit and re-worn as directed by the physician. The motorized closure device 1300 may include an alarm that audibly reminds the user to refit and tension the brace.

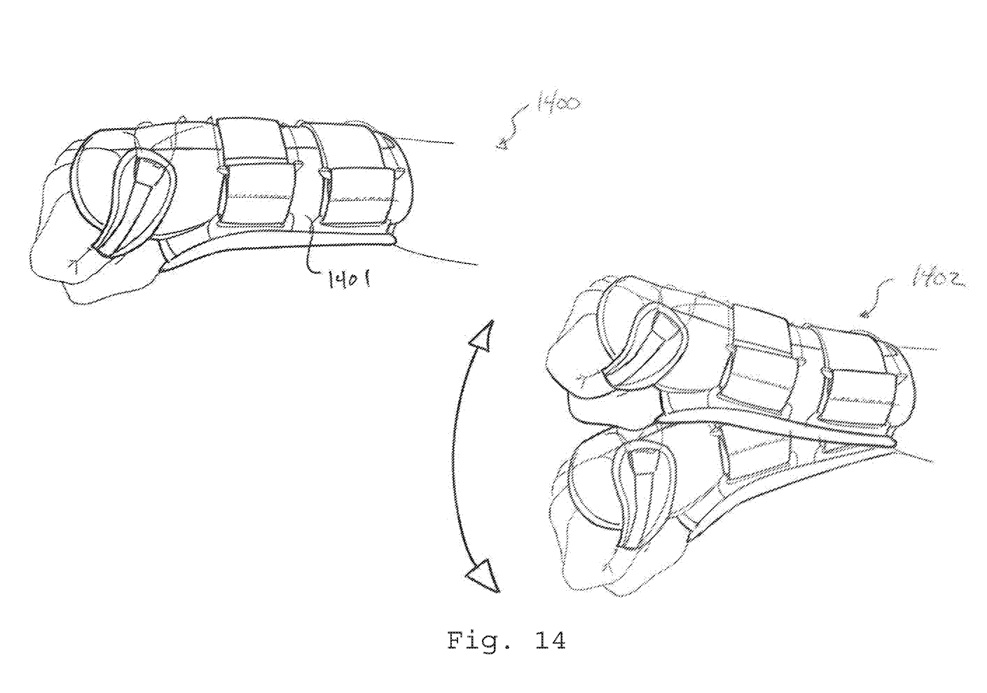

FIG. 14 illustrates a brace 1401 being used to provide physical therapy. The stiffness of the brace 1401 may be varied as described herein (i.e., via removable stays, adjustable lace tensions—motorized or manual, and the like) to allow the patient to flex the body part 1402 and perform physical therapy. In this manner, the brace 1401 functions as a therapy tool to promote recovery rather than merely preventing motion of the body part.

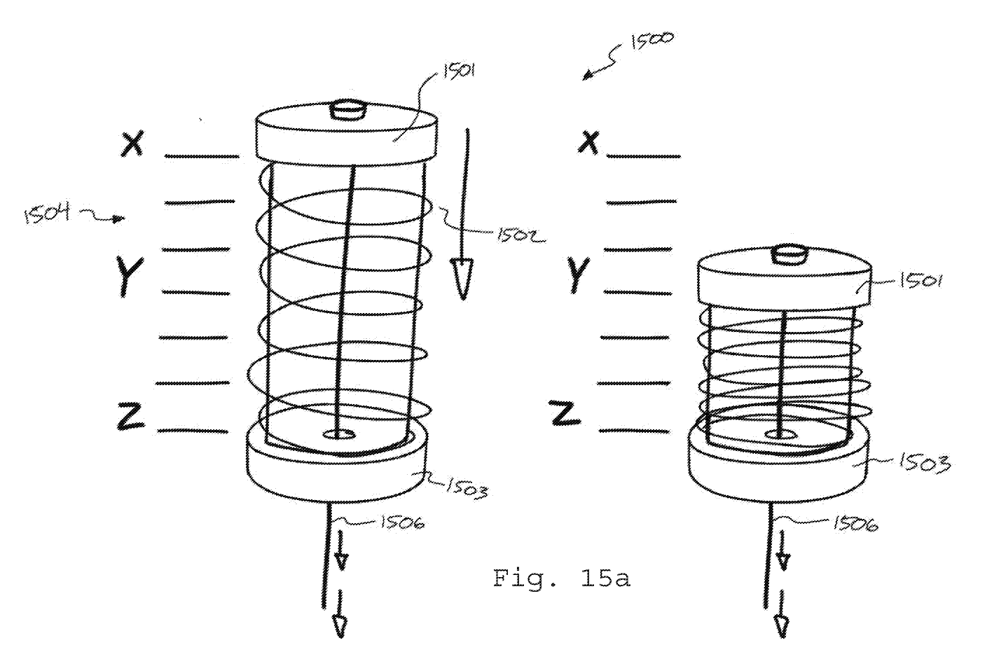

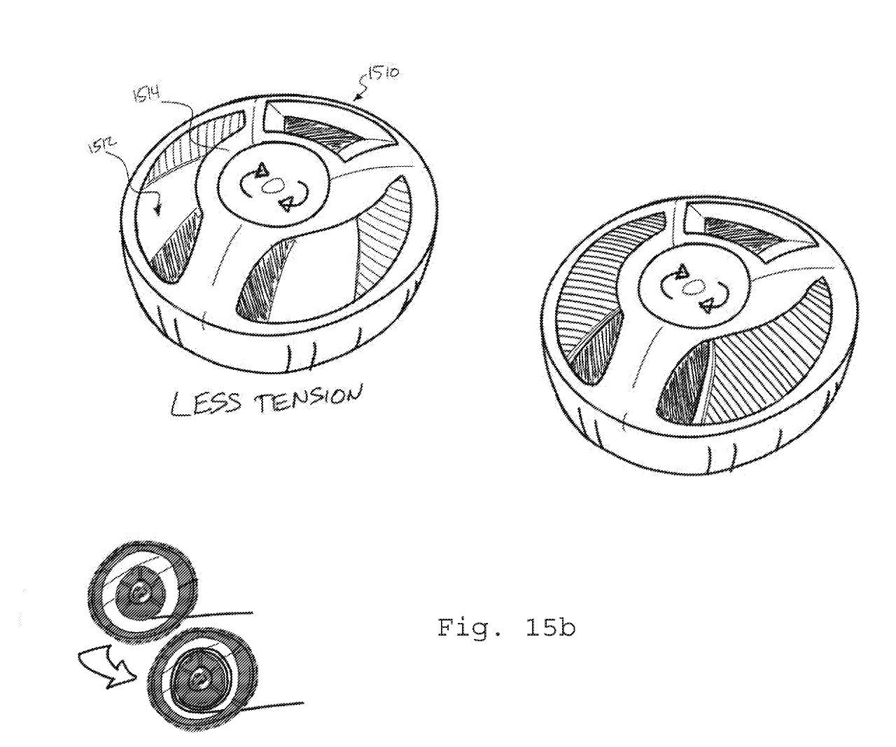

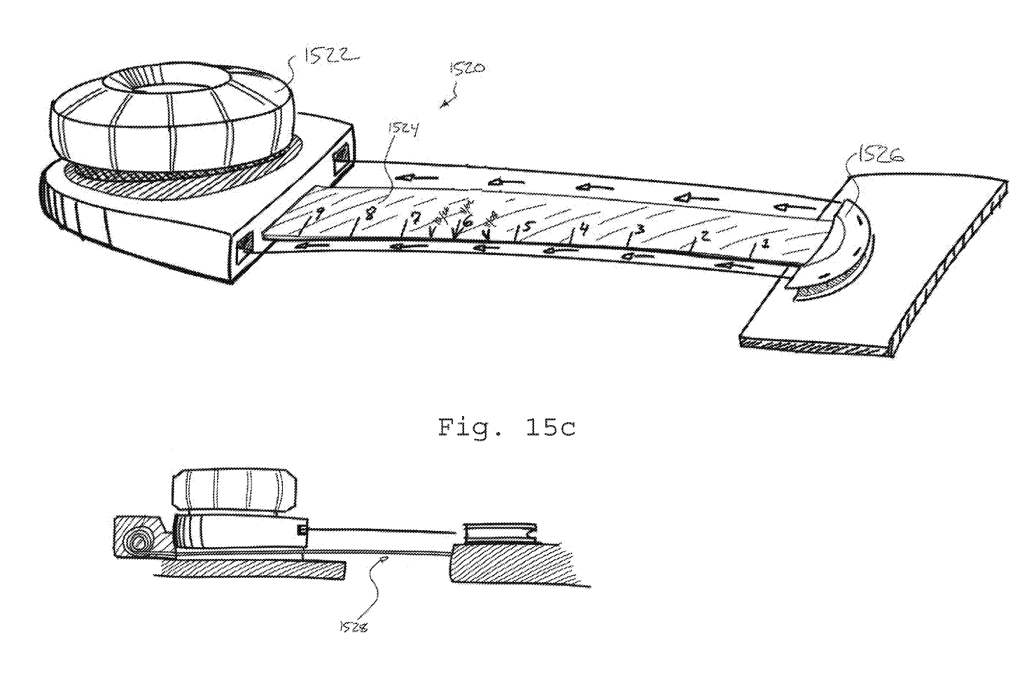

As briefly described above, in some embodiments it may be desirable to indicate the level of lace tension applied. This may be desirable to prevent a patient and/or a physician from overly tightening or loosening lace. FIG. 15a illustrates an indicator 1500 that may be used to visually indicate the lace tension applied. Indicator 1500 includes a housing 1504 having a top component 1501 that is coupled at an end of the lace 1506. A spring 1502 is coupled between the top component 1501 and a bottom component 1503. The bottom component 1503 is fixedly coupled with a brace or the reel system so that the bottom component does not move relative to the brace or reel system. As the lace is tensioned, the top component 1501 is pulled axially downward relative to the bottom component 1503, which compresses spring 1502. The housing 1504 includes indicia that visually indicate the tension applied to the lace. The indicia may indicate a level of tension that is too great or too little, such as by using red and green markings or any other color or markings. In this manner, the physician or the patient may tension the lace within a prescribed range.

FIG. 15b illustrates another embodiment 1510 of indicating a tension level. Specifically, the tension indicator is within the dial assembly and specifically underneath a window 1512 of the dial 1514. As the dial 1514 is rotated and tension applied to the lace, the tension level could be displayed through window 1512. Tension display may occur via a disc or component that is coupled with the spool and that is visible through the window 1512. The disc or component could be a spring that is forced to rotate as lace tension increases. As the disc or component rotates, visual indicia could be viewed through the window 1512, such as red or green coloring that indicates a prescribed or overly tight/loose lace tension. In another embodiment, the amount of lace that is wound around the spool may be visible through the window 1512. The window may include markings corresponding to the lace diameter wound around the spool. The marking may indicate levels of too much or too little lace tension.

FIG. 15c illustrates another embodiment 1520 of visually indicating lace tension. Specifically, a ruler 1524 could be coupled between the reel system 1522 and a guide 1526 to visually display a distance between a housing of the reel system 1522 and the guide 1526. The ruler 1524 may wind into a housing of the reel system 1522 and/or around a spool 1528 of the reel system as the brace is closed. The lace tension could be inferred based on the distance between the components. In addition, the physician could mark indicia on the ruler 1524 that instructs a patient on an amount of tension to apply to the lace over a defined time period based on a prescribed treatment plan. For example, the physician may mark 5 inches of separation between the housing and guide 1526 for a first week, 5.5 inches or separation for a second week, 6 inches of separation for a third week, and the like. FIG. 15d illustrates a similar embodiment 1530 except that marking 1536 are placed directly on the lace 1534. The markings may correspond to prescribed dosage or closure/tension levels as described herein. The physician or the patient may loosen or tighten lace tension until an appropriate marking 1536 is visible from housing 1532 of the reel system. The markings may be color coded or include other indicia as desired.

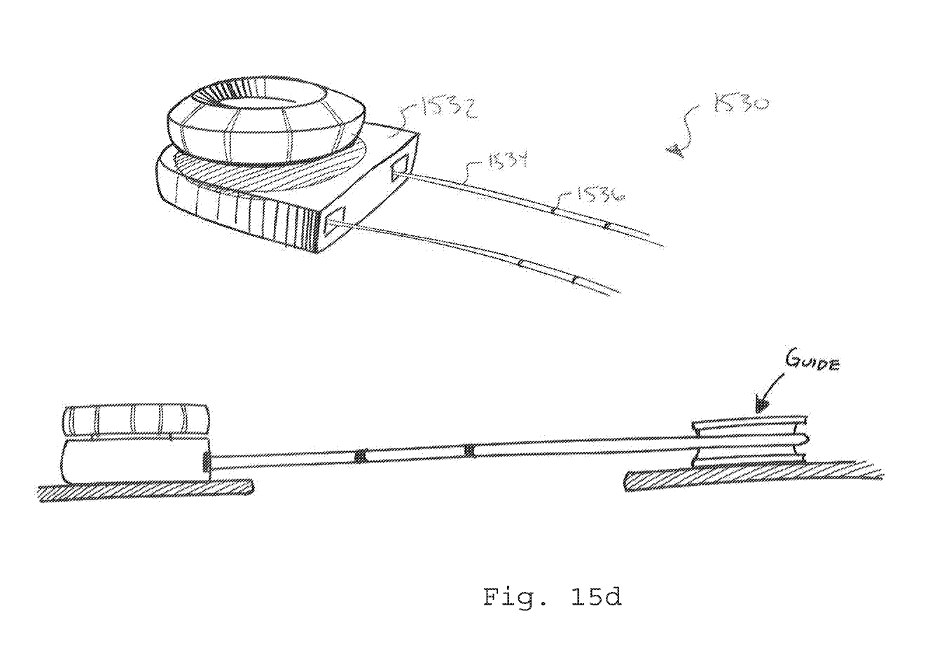

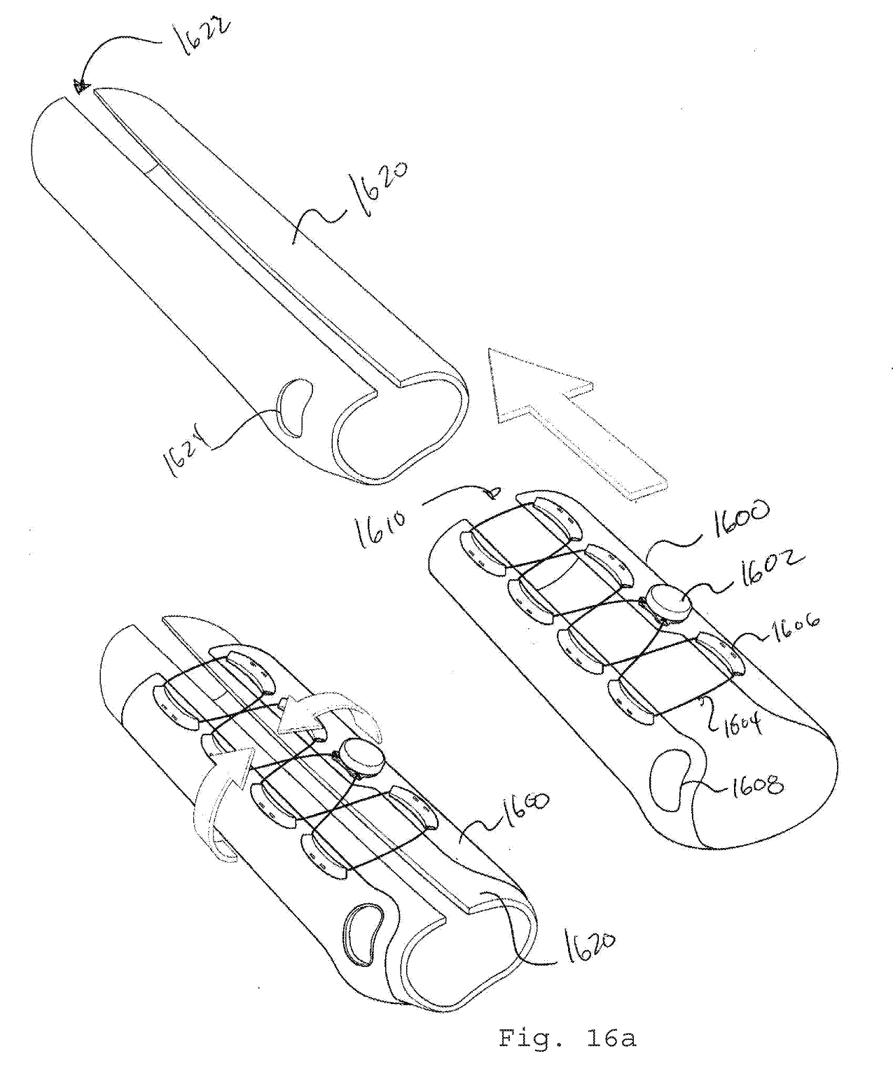

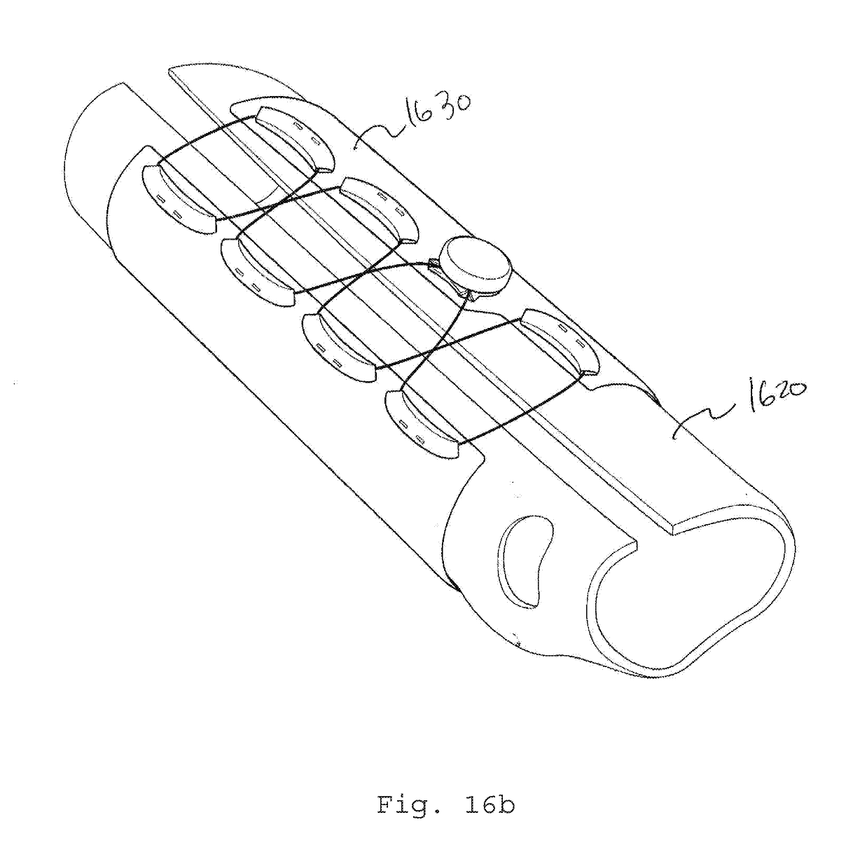

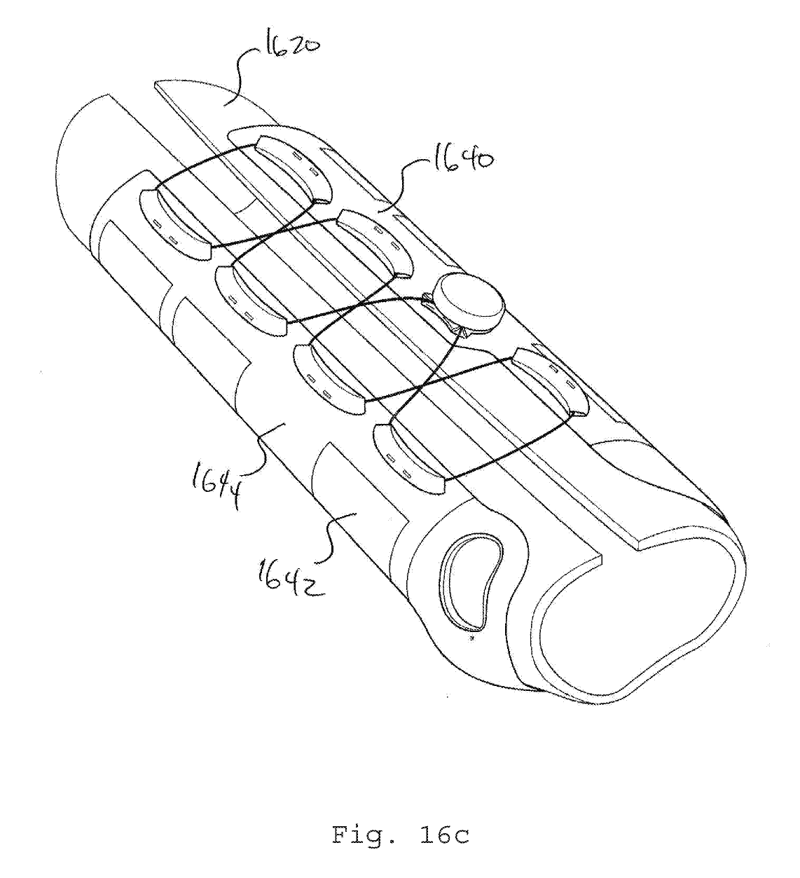

Referring now to FIG. 16a, illustrated is an embodiment of a sheath or sleeve 1600 that may be positioned over a bivalved cast 1620 and used to close or tighten the bivalved cast. The bivalved cast 1620 is a cast that is cut in half, or that include multiple cuts portions, along it longitudinal length (or elsewhere as required). The cut 1622, or multiple cuts, are designed to relieve pressure on a limb that is positioned within the cast 1620. The single longitudinal cut 1622, or multiple longitudinal cuts, divide the cast 1620 into two halves. The casts 1620 are often formed from layers of wrapped moistened gauze, such as gauze treated with fiberglass solutions and/or resins. The layers are allowed to dry and harden and then the cast 1620 is cut 1622. Traditional bivalve casts may be tightened about a limb by wrapping additional layers of often untreated gauze over the bivalve cast. Pressure may be relieved from the limb by unwrapping the gauze from the bivalve cast. Constant wrapping and unwrapping of the gauze from the bivalve cast is often burdensome and time consuming. Further, the use of gauze does not allow the tightness of the bivalve cast to be easily adjusted.

The sheath or sleeve 1600 (hereinafter sleeve 1600) eliminates the need to wrap and unwrap gauze in order to adjust the pressure of the cast 1620 about the limb. The sleeve 1600 is configured to fit over or atop the main body of the cast 1620 and apply a closure force or pressure to the cast 1620. This is achieved via a tightening mechanism 1602, which in the illustrated embodiment is a reel based device. The tightening mechanism 1602 is operably coupled with one or more tension members or lace 1604 that are guided along and across an opening 1610 of the sleeve 1600. Operation of the tightening mechanism 1602 (i.e., rotation of a knob member of the reel based device) effects tensioning of the lace 1604, such as by winding the lace 1604 about a spool that is positioned within a housing of the reel based device. Tensioning of the lace 1604 cases the opposing sides of the opening 1610 to be pulled toward one another.

As illustrated in FIG. 16a, the cast 1620 may be inserted within the sleeve 1600, such as by sliding the sleeve 1600 longitudinally over the main body of the cast 1620. With the sleeve 1600 positioned over the cast 1620, operation of the tightening mechanism 1602 causes the sleeve 1600 to constrict about the main body of the cast 1620, which causes the cast 1620 to tighten or constrict about the limb positioned within the cast 1620. In this manner, the sleeve 1600 may be used to increase the pressure on the limb, such as in response to atrophying of the limb or various other reasons.

The tightening mechanism 1602 may also be used to loosen the lace 1604, which releases the pressure that the sleeve 1600 exerts on the main body of the cast 1620 and relieves or reduces the pressure that the cast 1620 exerts on the limb. In this manner, the sleeve 1600 may be used to decrease the pressure on the limb, such as in response to the limb experiencing edema or other various issues. In some instances, the sleeve 1600 may be removed from the cast. In other instances, the sleeve may be fixedly connected to the cast, or otherwise non-removable from the cast 1620. For example, a physician may position the sleeve 1600 about the cast 1620 and then employ one of the reel devices described herein that prevent the user from fully removing the sleeve 1600, while allowing the user to adjust the lace tension to provide added comfort.

The cast 1620 and sleeve 1600 may have corresponding apertures, 1608 and 1624, or other features that enable the sleeve 1600 to be used with the cast 1620. For example, the cast 1620 and sleeve 1600 may include apertures, recessed portions, and the like to accommodate appendages (e.g., fingers) and/or components or features that are attached to the cast.

Referring now to FIG. 16b, illustrated is an embodiment of a sleeve 1630 that is positioned about the cast 1620. As illustrated, the sleeve 1630 may be axially or longitudinally shorter than the cast 1620. In some instances, the sleeve 1630 may only be positioned about or over an area of concern where pressure needs to be exerted on the limb. In other embodiments, multiple sleeves (not shown) may be positioned about the cast 1620. The multiple sleeves may be arranged to provide a desired pressure profile to the underlying limb.

In some instances, the cast may be made of a fabric or cloth material. The fabric or cloth material may have a very limited amount of stretch so that most of the force exerted on the sleeve via the tightening mechanism is transferred or applied to the main body of the cast 1620. The fabric or cloth material may be relatively flexible to enable a user or physician to easily open the sleeve and slide it over the cast 1620. In some instances, the sleeve may include stiffening components (not shown), or stays, that stiffen one or more areas of the sleeve and underlying cast 1620.

Referring now to FIG. 16c, in some embodiments the sleeve 1640 may include one or more tension or force directors 1642. The force directors 1642 may be designed to apply a desired constrictive force about the cast 1620. For example, the force directors 1642 may be areas of the sleeve 1640 that are stiffened, less elastic, or more rigid that a main body 1644 of the sleeve 1640. A compressive or constrictive force may be greater near the force directors 1642 due to the relatively rigid material. In a specific embodiment, the force directors 1642 may be made via applying a material (e.g., poly propylene and the like) atop the main body 1644 to form one or more strips or panels. In other embodiments, the force directors 1642 may be areas of increased flexibility or elasticity compared with the main body 1644. The increased flexibility may decrease the pressure that is applied immediately adjacent the force directors 1642. The force directors 1642 may be built into the main body 1644 by varying the weave pattern of the fabric or integrating one or more materials into the weave, or may be achieved by attaching one or more separate components to the main body 1644. In some instances, the force directors 1642 may be areas where the main body 1644 is cut out or removed.

The sleeves described herein represent components that are typically separate from the bivalve cast and that are positionable over the casts to adjust the fit of the cast about an underlying limb. The sleeves greatly reduce or eliminate many of the issues with wearing bivalve casts, such as wrapping and unwrapping gauze. The sleeves also enable a user or physician to quickly and conveniently adjust the pressure that the cast exerts on the underlying limb.

Having described several embodiments, it will be recognized by those of skill in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included.

As used herein and in the appended claims, the singular forms “a”, “an”, and “the” include plural referents unless the context clearly dictates otherwise. Thus, for example, reference to “a process” includes a plurality of such processes and reference to “the device” includes reference to one or more devices and equivalents thereof known to those skilled in the art, and so forth.

Also, the words “comprise,” “comprising,” “include,” “including,” and “includes” when used in this specification and in the following claims are intended to specify the presence of stated features, integers, components, or steps, but they do not preclude the presence or addition of one or more other features, integers, components, steps, acts, or groups.

What is claimed is:

1. A device for tightening an article about a limb comprising:
a tension member that is guided about the article and tensionable to close or tighten the article about the limb;
a tightening mechanism that is configured to tension the tension member to close or tighten the article, and that is further configured to maintain the tension of the tension member so that the article remains closed or tightened about the limb; and
a tension limiting mechanism that prevents loosening of the tension member's tension beyond a determined threshold such that the article remains closed or tightened about the limb.

2. The device according to claim 1, wherein the tension limiting mechanism is operably coupled with a rotatable knob of the tightening mechanism, the tension limiting mechanism being engageable with a stop member to prevent rotation of the knob.

3. The device according to claim 2, wherein the tension limiting mechanism is a boss or post and the stop member is a channel within which the boss or post is disposed.

4. The device according to claim 1, wherein the tension limiting mechanism is an additional tension member that is windable about a spool component of the tightening mechanism, the additional tension member being configured to prevent rotation of the spool component after a defined amount of the additional tension member is wound about the spool component.

5. The device according to claim 1, wherein the tension limiting mechanism comprises a crimp member that is attached to the tension member, the crimp member being engagable with a stop member after a defined amount of the tension member is wound about a spool component of the tightening mechanism to prevent rotation of the spool component.

6. The device according to claim 1, wherein the tightening mechanism comprises a reel based device that is operably coupled with the tension member so that operation of the tightening mechanism tensions the tension member, and wherein the tension limiting mechanism is coupled with a portion of the tension member that is remotely positioned from the tightening mechanism.

7. A method for tightening an article about a limb comprising:
providing an article with a device that is configured to tighten the article about the limb, the device including:
a tension member that is guided about the article;
a tightening mechanism that is configured to tension the tension member and to maintain the tension of the tension member; and
a tension limiting mechanism that prevents loosening of the tension member's tension article beyond a determined threshold;
placing the article about the limb;
tensioning the tension member via the tightening mechanism to close or tighten the article about the limb; and
restricting loosening of the tension member's tension via the tension limiting mechanism so that the article remains closed or tightened about the limb.

8. The method of claim 7, wherein the tension limiting mechanism is operably coupled with a rotatable knob of the tightening mechanism, the tension limiting mechanism being engageable with a stop member to prevent rotation of the knob.

9. The method of claim 8, wherein the tension limiting mechanism is a boss or post and the stop member is a channel within which the boss or post is disposed.

10. The method of claim 7, wherein the tension limiting mechanism is an additional tension member that is windable about a spool component of the tightening mechanism, the additional tension member being configured to prevent rotation of the spool component after a defined amount of the additional tension member is wound about the spool component.

11. The method of claim 7, wherein the tension limiting mechanism comprises a crimp member that is attached to the tension member, the crimp member being engagable with a stop member after a defined amount of the tension member is wound about a spool component of the tightening mechanism to prevent rotation of the spool component.

12. The method of claim 7, wherein the tightening mechanism comprises a reel based device that is operably coupled with the tension member so that operation of the tightening mechanism tensions the tension member, and wherein the tension limiting mechanism is coupled with a portion of the tension member that is remotely positioned from the tightening mechanism.
